SEQUENCE LISTING (133 sequences)

SEQ ID NO: 1 - protein, 450 aa, Streptococcus pneumoniae
MGKYFGTDGVRGEANLELTPELAFKLGRFGGYVLSQHETEAPKVFVGRDTRISGEMLESALVAGLLSVGIHVYKLGVLATPAVAYLVETEGASAGVMISASHNPALDNGIKFFGGDGFKLDDEKEAEIEALLDAEEDTLPRPSAEGLGILVDYPEGLRKYEGYLVSTGTPLDGMKVALDTANGAASTSARQIFADLGAQLTVIGETPDGLNINLNVGSTHPEALQEVVKESGSAIGLAFDGDSDRLIAVDENGDIVDGDKIMYIIGKYLSEKGQLAQNTIVTTVMSNLGFHKALNREGINKAVTAVGDRYVVEEMRKSGYNLGGEQSGHVILMDYNTTGDGQLSAVQLTKIMKETGKSLSELAAEVTIYPQKLVNIRVENVMKEKAMEVPAIKAIIEKMEEEMAGNGRILVRPSGTEPLLRVMAEAPTTEEVNYYVDTITDVVRAEIGID

SEQ ID NO: 2 - protein, 693 aa, Streptococcus pneumoniae
MAREFSLEKTRNIGIMAHVDAGKTTTTERILYYTGKIHKIGETHEGASQMDWMEQEQERGITITSAATTAQWNNHRVNIIDTPGHVDFTIEVQRSLRVLDGAVTVLDSQSGVEPQTETVWRQATEYGVPRIVFANKMDKIGADFLYSVSTLHDRLQANAHPIQLPIGSEDDFRGIIDLIKMKAEIYTNDLGTDILEEDIPAEYLDQAQEYREKLIEAVAETDEELMMKYLEGEEITNEELKAGIRKATINVEFFPVLCGSAFKNKGVQLMLDAVIDYLPSPLDIPAIKGINPDTDAEEIRPASDEEPFAALAFKIMTDPFVGRLTFFRVYSGVLQSGSYVLNTSKGKRERIGRILQMHANSRQEIDTVYSGDIAAAVGLKDTTTGDSLTDEKAKIILESINVPEPVIQLMVEPKSKADQDKMGIALQKLAEEDPTFRVETNVETGETVISGMGELHLDVLVDRMRREFKVEANVGAPQVSYRETFRASTQARGFFKRQSGGKGQFGDVWIEFTPNEEGKGFEFENAIVGGVVPREFIPAVEKGLVESMANGVLAGYPMVDVKAKLYDGSYHDVDSSETAFKIAASLSLKEAAKSAQPAILEPMMLVTITVPEENLGDVMGHVTARRGRVDGMEAHGNSQIVRAYVPLAEMFGYATVLRSASQGRGTFMMVFDHYEDVPKSVQEEIIKKNKGED

SEQ ID NO: 3 - protein, 100 aa, Streptococcus pneumoniae
MKITQEEVTHVANLSKLRFSEEETAAFATTLSKIVDMVELLGEVDTTGVAPTTTMADRKTVLRPDVAEEGIDRDRLFKNVPEKDNYYIKVPAILDNGGDA

SEQ ID NO: 4 - protein, 328 aa, Streptococcus pneumoniae
MTSTKQHKKVILVGDGAVGSSYAFALVNQGIAQELGIIEIPQLHEKAVGDALDLSHALAFTSPKKIYAAQYSDCADADLVVITAGAPQKPGETRLDLVGKNLAINKSIVTQVVESGFKGIFLVAANPVDVLTYSTWKFSGFPKERVIGSGTSLDSARFRQALAEKLDVDARSVHAYIMGEHGDSEFAVWSHANIAGVNLEEFLKDTQNVQEAELIELFEGVRDAAYTIINKKGATYYGIAVALARITKAILDDENAVLPLSVFQEGQYGVENVFIGQPAVVGAHGIVRPVNIPLNDAETQKMQASAKELQAIIDEAWKNPEFQEASKN

SEQ ID NO: 5 - protein, 335 aa, Streptococcus pneumoniae
MVVKVGINGFGRIGRLAFRRIQNVEGVEVTRINDLTDPVMLAHLLKYDTTQGRFDGTVEVKEGGFEVNGKFIKVSAERDPEQIDWATDGVEIVLEATGFFAKKEAAEKHLKGGAKKVVITAPGGNDVKTVVFNTNHDVLDGTETVISGASCTTNCLAPMAKALQDNFGVVEGLMTTIHAYTGDQMILDGPHRGGDLRRARAGAANIVPNSTGAAKAIGLVIPELNGKLDGSAQRVPTPTGSVTELVAVLEKNVTVDEVNAAMKAASNESYGYTEDPIVSSDIVGMSYGSLFDATQTKVLDVDGKQLVKVVSWYDNEMSYTAQLVRTLEYFAKIAK

SEQ ID NO: 6 - protein, 336 aa, Streptococcus pneumoniae
MAILVTGGAGYIGSHTVVELLNLGKEVIIVDNLSNSSILVLDRIEAITGIRPVFYELDVCDKQALRKVFEQESIDAAIHFAGYKAVGESVQKPVMYYKNNIMSTLALVEVMSEFNVKKIVFSSSATVYGINNQSPLIETMQTSATNPYGYTKVMLEQILKDVHVADSEWSIALLRYFNPIGAHESGLIGEDPSGIPNNLMPYIAQVAVGKLSELSVFGNDYDTLDGTGVRDYIHVVDLAIGHIKALEKVSEKTDVYIYNLGSGEGTSVLQLVNTFESVNKIPIPYKIVPRRSGDVATCYANADKAYKELNWRTTKSIEDMCRDTWNWQSKNPNGYN

SEQ ID NO: 7 - protein, 620 aa, Streptococcus pneumoniae
MNIIEEIMTKLREDIRNIAIIAHVDHGKTTLVDELLKQSETLDARTELAERAMDSNDIEKERGITILAKNTAVAYNGTRINIMDTPGHADFGGEVERIMKMVDGVVLVVDAYEGTMPQTRFVLKKALEQDLVPIVVVNKIDKPSARPAEVVDEVLELFIELGADDDQLDFPVVYASAINGTSSLSDDPADQEATMAPIFDTIIDHIPAPVDNSDEPLQFQVSLLDYNDFVGRIGIGRVFRGTVKVGDQVTLSKLDGTTKNFRVTKLFGFFGLERREIQEAKAGDLIAVSGMEDIFVGETITPTDAVEALPILHIDEPTLQMTFLVNNSPFAGKEGKWVTSRKVEERLQAELQTDVSLRVDPTDSPDKWTVSGRGELHLSILIETMRREGYELQVSRPEVIVKEIDGVKCEPFERVQIDTPEEYQGSVIQSLSERKGEMLDMISTGNGQTRLVFLVPARGLIGYSTEFLSMTRGYGIMNHTFDQYLPLIPGEIGGRHRGALVSIDAGKATTYSIMSIEERGTIFVNPGTEVYEGMIIGENSRENDLTVNITKAKQMTNVRSATKDQTAVIKTPRILTLEESLEFLNDDEYMEVTPESIRLRKQILNKAEREKANKKKKSAE

SEQ ID NO: 8 - protein, 520 aa, Streptococcus pneumoniae
MSNISTDLQDVEKIIVLDYGSQYNQLISRRIREIGVFSELKSHKISAAEVREVNPVGIILSGGPNSVYEDGSFDIDPEIFELGIPILGICYGMQLLTHKLGGKVVPAGDAGNREYGQSTLTHTPSALFESTPDEQTVLMSHGDAVTEIPADFVRTGTSADCPYAAIENPDKHIYGIQFHPEVRHSVYGNDILRNFALNICKAKGDWSMDNFIDMQIKKIRETVGDKRVLLGLSGGVDSSVVGVLLQKAIGDQLICIFVDHGLLRKGEADQVMDMLGGKFGLNIVKADAAKRFLDKLAGVSDPEQKRKIIGNEFVYVFDDEASKLKDVKFLAQGTLYTDVIESGTDTAQTIKSHHNVGGLPEDMQFELIEPLNTLYKDEVRALGTELGMPDHIVWRQPFPGPGLAIRVMGEITEEKLETVRESDAILREEIAKAGLDRDIWQYFTVNTGVRSVGVMGDGRTYDYTIAIRAITSIDGMTADFAKIPWEVLQKISVRIVNEVDHVNRIVYDITSKPPATVEWE

SEQ ID NO: 9 - protein, 448 aa, Streptococcus pneumoniae
MTSAKEYIQSVFETVKARNGHEAEFLQAVEEFFNTLEPVFEKHPEYIEENILARITEPERVVSFRVPWVDRDGKIQVNRGYRVQFNSAVGPYKGGLRFHPTVNQGILKFLGFEQIFKNVLTGLPIGGGKGGSDFDPKGKTDAEVMRFCQSFMTELQKHIGPSLDVPAGDIGVGGREIGYLYGQYKRLNQFDAGVLTGKPLGFGGSLIRPEATGYGLVYYTEEMLKANGNSFAGKKVVISGSGNVAQYALQKATELGATVISVSDSNGYVIDENGIDFDLLVDVKEKRRARLTEYAAEKATATYHEGTVWTYAGNYDIALPCATQNEINGEAAKRLVAQGVICVSEGANMPSDLDAIKVYKENGIFYGPAKAANAGGVAVSALEMSQNSLRLSWTREEVDGRLKDIMTNIFNTAKTTSETYGLDKDYLAGANIAAFENVANAMIAQGIV

SEQ ID NO: 10 - protein, 346 aa, Streptococcus pneumoniae
MAEITAKLVKELREKSGAGVMDAKKALVETDGDIEKAIELLREKGMAKAAKKADRVAAEGLTGVYVNGNVAAVIEVNAETDFVAKNAQFVELVNTTAKVIAEGKPANNEEALALIMPSGETLEAAYVSATATIGEKISFRRFALIEKTDAQHFGAYQHNGGRIGVISVVEGGDEALAKQLSMHIAAMKPTVLSYKELDEQFVKDELAQLNHVIDQDNESRAMVNKPALPHLKYGSKAQLTDDVIAQAEADIKAELAAEGKPEKIWDKIIPGKMDRFMLDNTKVDQAYTLLAQVYIMDDSKTVEAYLESVNASVVEFARFEVGEGIEKAANDFEAEVAATMAAALNN

SEQ ID NO: 11 - protein, 398 aa, Streptococcus pneumoniae
MAKLTVKDVDLKGKKVLVRVDFNVPLKDGVITNDNRITAALPTIKYIIEQGGRAILFSHLGRVKEEADKAGKSLAPVAADLAAKLGQDVVFPGVTRGAELEAAINALEDGQVLLVENTRYEDVDGKKESKNDPELGKYWASLGDGIFVNDAFGTAHRAHASNVGISANVEKAVAGFLLENEIAYIQEAVETPERPFVAILGGSKVSDKIGVIENLLEKADNVLIGGGMTYTFYKAQGIEIGNSLVEEDKLDVAKALLEKANGKLILPVDSKEANAFAGYTEVRDTEGEAVSEGFLGLDIGPKSIAKFDEALTGAKTVVWNGPMGVFENPDFQAGTIGVMDAIVKQPGVKSIIGGGDSAAAAINLGRADKFSWISTGGGASMELLEGKVLPGLAALTEK

SEQ ID NO: 12 - protein, 400 aa, Streptococcus pneumoniae
MNEFEDLLNSVSQVETGDVVSAEVLTVDATQANVAISGTGVEGVLTLRELTNDRDADINDFVKVGEVLDVLVLRQVVGKDTDTVTYLVSKKRLEARKAWDKLVGREEEVVTVKGTRAVKGGLSVEFEGVRGFIPASMLDTRFVRNAERFVGQEFDTKIKEVNAKENRFILSRREVVEAATAAARAEVFGKLAVGDVVTGKVARITSFGAFVDLGGVDGLVHLTELSHERNVSPKSVVTVGEEIEVKILDLNEEEGRVSLSLKATVPGPWDGVEQKLAKGDVVEGTVKRLTDFGAFVEVLPGIDGLVHVSQISHKRIENPKEALKVGQEVQVKVLEVNADAERVSLSIKALEERPAQEEGQKEEKRAARPRRPRRQEKRDFELPETQTGFSMADLFGDIEL

SEQ ID NO: 13 - protein, 474 aa, Streptococcus pneumoniae
MTKANFGVVGMAVMGRNLALNIESRGYTVAIYNRSKEKTEDVIACHPEKNFVPSYDVESFVNSIEKPRRIMLMVQAGPGTDATIQALLPHLDKGDILIDGGNTFYKDTIRRNEELANSGINFIGTGVSGGEKGALEGPSIMPGGQKEAYELVADVLEEISAKAPEDGKPCVTYIGPDGAGHYVKMVHNGIEYGDMQLIAESYDLMQHLLGLSAEDMAEIFTEWNKGELDSYLIEITADILSRKDDEGQDGPIVDYILDAAGNKGTGKWTSQSSLDLGVPLSLITESVFARYISTYKEERVHASKVLPKPAAFNFEGDKAELIEKIRQALYFSKIISYAQGFAQLRVASKENNWNLPFADIASIWRDGCIIRSRFLQKITDAYNRDADLANLLLDEYFLDVTAKYQQAVRDIVALAVQAGVPVPTFSAAITYFDSYRSADLPANLIQAQRDYFGAHTYQRKDKEGTFHYSWYDEK

SEQ ID NO: 14 - protein, 444 aa, Streptococcus pneumoniae
MNAIQESFTDKLFANYEANVKYQAIENAASHNGIFAALERRQSHVDNTPVFSLDLTKDKVTNQKASGRCWMFAALNTFRHKLISQYKLENFELSQAHTFFWDKYEKSNWFLEQVIATSDQELTSRKVSFLLQTPQQDGGQWDMVVSLFEKYGVVPKSVYPESVSSSSSRELNAILNKLLRQDAQILRDLLVSGADQATVQAKKEDLLQEIFNFLAMSLGLPPRKFDFAYRDKDNNYKSEKGITPQEFYKKYVNLPLEDYVSVINAPTADKPYGKSYTVEMLGNVVGSRAVRYINVPMERLKELAIAQMQAGETVWFGSDVGQLSNRKAGILATDVYDFESSMDIKLTQDKAGRLDYSESLMTHAMVLTGVDLDENGKSTKWKVENSWGDKVGTDGYFVASDAWMDEYTYQIVVRKELLTAEEQAAYGAEPIVLAPWDPMGALAE

SEQ ID NO: 15 - protein, 1058 aa, Streptococcus pneumoniae
MPKRTDIQKIMVIGSGPIIIGQAAEFDYAGTQACLSLKEEGYEVVLVNSNPATIMTDKEIADKVYIEPITLEFVTRILRKEGPDALLPTLGGQTGLNMAMELSKNGILDELGVELLGTKLSAIDQAEDRDLFKQLMEELEQPIPESEIVNTVEEAVAFAATIGYPVIVRPAFTLGGTGGGMCANEKELREITENGLKLSPVTQCLIERSIAGFKEIEYEVMRDSADNALVVCNMENFDPVGIHTGDSIVFAPAQTMSDYENQMLRDASLSIIRALKIEGGCNVQLALDPNSFKYYVIEVNPRVSRSSALASKATGYPIAKLAAKIAVGLTLDEVINPVTGSTYAMFEPALDYVVAKIPRFPFDKFEKGERRLGTQMKATGEVMAIGRNIEESLLKACRSLEIGVHHNEIPELAAVSDDALIEKVVKAQDDRLFYVSEAIRRGYTPEEIAELTKIDIFYLDKLLHIFEIEQELGAHPQDLEVLKTAKLNGFSDRKIAELWGTTDDKVRQLRLENKIVPVYKMVDTCAAEFDSETPYFYSTYGWENESIRSDKESVLVLGSGPIRIGQGVEFDYATVHSVKAIQAAGYEAIIMNSNPETVSTDFSVSDKLYFEPLTFEDVMNVIDLEQPKGVIVQFGGQTAINLAEPLAKAGVTILGTQVADLDRAEDRDLFEQALKELDIPQPPGQTATNEEEAALAARKIGFPVLVRPSYVLGGRAMEIVENEEDLRSYMRTAVKASPDHPVLVDSYIVGQECEVDAISDGKNVLIPGIMEHIERAGVHSGDSMAVYPPQTLSQKVQETIADYTKRLAIGLHCLGMMNIQFVIKDEKVYVIEVNPRASRTVPFLSKVTNIPMAQVATKLILGQSLSELGYQNGLYPESTRVHIKAPVFSFTKLAKVDSLLGPEMKSTGEVMGSDATLEKALYKAFEASYLHLPTFGNVVFTIADDAKEEALNLARRFQNIGYGILATEGTAAFFASHGLQAQPVGKIGDDDKDIPSFVRKGRIQAIINTVGTKRTADEDGEQIRRSAIEHGVPLFTALDTANAMLKVLESRSFVTEAI

SEQ ID NO: 16 - protein, 332 aa, Streptococcus pneumoniae
MTIMSIGIIIASHGEFAAGIHQSGSMIFGEQEKVQVVTFMPNEGPDDLYAKFNNAVAAFDAEDEVLVLADLWSGSPFNQASRVMGENPERKFAIITGLNLPMLIQAYTERLMDAAAGVEKVAANIIKEAKDGIKALPEELNPVEEVASAAAAPVAQTAIPEGTVIGDGKLKINLARLDTRLLHGQVATAWTPDSKANRIIVASDNVAKDDLRKELIKQAAPGNVKANVVPIQKLIEISKDPRFGETHALILFETPQDALRAIEGGVPIKTLNVGSMAHSTGKTLVNTVLSMDKEDVATFEKMRDLGVEFDVRKVPNDSKKDLFDLINKANVK

SEQ ID NO: 17 - protein, 259 aa, Streptococcus pneumoniae
MAVISMKQLLEAGVHFGHQTRRWNPKMAKYIFTERNGIHVIDLQQTVKYADQAYDFMRDAAANDAVVLFVGTKKQAADAVAEEAVRSGQYFINHRWLGGTLTNWGTIQKRIARLKEIKRMEEDGTFEVLPKKEVALLNKQRARLEKFLGGIEDMPRIPDVMYVVDPHKEQIAVKEAKKLGIPVVAMVDTNTDPDDIDVIIPANDDAIRAVKLITAKLADAIIEGRQGEDAVAVEAEFAALETQADSIEEIVEVVEGDNA

SEQ ID NO: 18 - protein, 312 aa, Streptococcus pneumoniae
MTTNRLQVSLPGLDLKNPIIPASGCFGFGQEYAKYYDLNLLGSIMIKATTLEPRFGNPTPRVAETPAGMLNAIGLQNPGLEVVLAEKLPWLEREYPNLPIIANVAGFSKQEYAAVSHGISKATNVKAIELNISCPNVDHCNHGLLIGQDPDLAYDVVKAAVEASEVPVYVKLTPSVTDIVTVAKAAEDAGASGLTMINTLVGMRFDLKTRKPILANGTGGMSGPAVFPVALKLIRQVAQTTDLPIIGMGGVDSTEAALEMYLAGASAIGVGTANFTNPYACPDIIENLPKVMDKYGISSLEELRQEVKESLR

SEQ ID NO: 19 - protein, 307 aa, Streptococcus pneumoniae
MSENQQALNHVVSMEDLTVDQVMKLIKRGIEFKNGAQLPYEDHPIVSNLFFEDSTRTHKSFEVAEIKLGLERLDFDVKTSSVNKGETLYDTILTLSALGVDVCVIRHPEVDYYRELIASPTITTSIINGGDGSGQHPSQSLLDLMTIYEEFGHFEGLKVAIAGDLDHSRVAKSNMQILKRLGAELFFAGPEEWRSQEFADYGQFVTIDEIIDQVDVMMFLRVQHERHDSGAVFSKEDYHAQHGLTQERYDRLKETAILMHPAPINRDVEIADHLVEAPKSRIVQQMTNGVFVRMAILESVLASRNAN

SEQ ID NO: 20 - protein, 398 aa, Streptococcus pneumoniae
MAKEKYDRSKPHVNIGTIGHVDHGKTTLTAAITTVLARRLPSSVNQPKDYASIDAAPEERERGITINTAHVEYETEKRHYAHIDAPGHADYVKNMITGAAQMDGAILVVASTDGPMPQTREHILLSRQVGVKHLIVFMNKVDLVDDEELLELVEMEIRDLLSEYDFPGDDLPVIQGSALKALEGDSKYEDIVMELMNTVDEYIPEPERDTDKPLLLPVEDVFSITGRGTVASGRIDRGIVKVNDEIEIVGIKEETQKAVVTGVEMFRKQLDEGLAGDNVGVLLRGVQRDEIERGQVIAKPGSINPHTKFKGEVYILTKEEGGRHTPFFNNYRPQFYFRTTDVTGSIELPAGTEMVMPGDNVTIDVELIHPIAVEQGTTFSIREGGRTVGSGMVTEIEA

SEQ ID NO: 21 - protein, 46 aa, Streptococcus pneumoniae
MKSTKEEIQTIKTLLKDSRTAKYHKRLQIVLFCLMGKSYKEIIELL

SEQ ID NO: 22 - protein, 398 aa, Streptococcus pneumoniae
MAKLTVKDVDLKGKKVLVRVDFNVPLKDGVITNDNRITAALPTIKYIIEQGGRAILFSHLGRVKEESDKAGKSLAPVAADLAAKLGQDVVFPGVTRGAELEAAINALEDGQVLLVENTRYEDVDGKKESKNDPELGKYWASLGDGIFVNDAFGTAHRAHASNVGISANVEKAVAGFLLENEIAYIQEAVETPERPFVAILGGSKVSDKIGVIENLLEKADKVLIGGGMTYTFYKAQGIEIGNSLVEEDKLDVAKALLEKANGKLILPVDSKEANAFAGYTEVRDTEGEAVSEGFLGLDIGPKSIAKFDEALTGAKTVVWNGPMGVFENPDFQAGTIGVMDAIVKQPGVKSIIGGGDSAAAAINLGRADKFSWISTGGGASMELLEGKVLPGLAALTEK

SEQ ID NO: 23 - protein, 276 aa, Streptococcus pneumoniae
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADLQQIMVRLSDGQFDYKIFDKIGVETVIKNQGLDNLKVIELPSDQQPYVYPLLAQGQDELKSFVDKRIKELYKDGTLEKLSKQFFGDTYLPAEADIK

SEQ ID NO: 24 - protein, 630 aa, Streptococcus pneumoniae
MTRYQDDFYDAINGEWQQTAEIPADKSQTGGFVDLDQEIEDLMLATTDKWLAGEEVPEDAILENFVKYHRLVRDFDKREADGITPVLPLLKEFQELETFADFTAKLAEFELAGKPNFLPFGVSPDFMDARINVLWASAPSTILPDTTYYAEEHPQREELLTLWKESSANLLKAYDFSDEEIEDLLEKRLELDRRVAAVVLSNEESSEYAKLYHPYSYEDFKKFAPALPLDDFFKAVIGQLPDKVIVDEERFWQAAEQFYSEEAWSLLKATLILSVVNLSTSYLTEDIRVLSGAYSRALSGVPEAKDKVKAAYHLAQEPFKQALGLWYAREKFSPEAKADVEKKVATMIDVYKERLLKNDWLTPETCKQAIVKLNVIKPYIGYPEELPARYKDKVVNETASLFENALAFARVEIKHSWSKWNQPVDYKEWGMPAHMVNAYYNPQKNLIVFPAAILQAPFYDLHQSSSANYGGIGAVIAHEISHAFDTNGASFDENGSLKDWWTESDYAAFKEKTQKVIDQFDGQDSYGATINGKLTVSENVADLGGIAAALEAAKREADFSAEEFFYNFGRIWRMKGRPEFMKLLASVDVHAPAKLRVNVQVPNFDDFFTTYDVKEGDGMWRSPEERVIIW

SEQ ID NO: 25 - protein, 149 aa, Streptococcus pneumoniae
MIGVVARENAAEQIKQYQKFTVNISDETSMLAMEQAGFISHQEKLERLGVHYEISERTQTPILDACPLVLDCRVDRIVEEDGICHIFAKILERLVAPELLDEKGHFKNQLFAPTYFMGDGYQRVYRYLDKRVDMKGSFIKKARKKDGKN

SEQ ID NO: 26 - protein, 17 aa, Artificial Sequence
  Synthetic
AVAYLVETEGASAGVMI

SEQ ID NO: 27 - protein, 12 aa, Artificial Sequence
  Synthetic
MKEKAMEVPAIK

SEQ ID NO: 28 - protein, 17 aa, Artificial Sequence
  Synthetic
YYVDTITDVVRAEIGID

SEQ ID NO: 29 - protein, 41 aa, Artificial Sequence
  Synthetic
ASHNPALDNGIKFFGGDGFKLDDEKEAEIEALLDAEEDTLP

SEQ ID NO: 30 - protein, 36 aa, Artificial Sequence
  Synthetic
IGLAFDGDSDRLIAVDENGDIVDGDKIMYIIGKYLS

SEQ ID NO: 31 - protein, 26 aa, Artificial Sequence
  Synthetic
TGKSLSELAAEVTIYPQKLVNIRVEN

SEQ ID NO: 32 - protein, 34 aa, Artificial Sequence
  Synthetic
IIEKMEEEMAGNGRILVRPSGTEPLLRVMAEAPT

SEQ ID NO: 33 - protein, 9 aa, Artificial Sequence
  Synthetic
TAQWNNHRV

SEQ ID NO: 34 - protein, 14 aa, Artificial Sequence
  Synthetic
LDIPAIKGINPDTD

SEQ ID NO: 35 - protein, 19 aa, Artificial Sequence
  Synthetic
GSYHDVDSSETAFKIAASL

SEQ ID NO: 36 - protein, 21 aa, Artificial Sequence
  Synthetic
HKIGETHEGASQMDWMEQEQE

SEQ ID NO: 37 - protein, 34 aa, Artificial Sequence
  Synthetic
LMMKYLEGEEITNEELKAGIRKATINVEFFPVLC

SEQ ID NO: 38 - protein, 10 aa, Artificial Sequence
  Synthetic
RDRLFKNVPE

SEQ ID NO: 39 - protein, 12 aa, Artificial Sequence
  Synthetic
DNYYIKVPAILD

SEQ ID NO: 40 - protein, 48 aa, Artificial Sequence
  Synthetic
ETAAFATTLSKIVDMVELLGEVDTTGVAPTTTMADRKTVLRPDVAEEG

SEQ ID NO: 41 - protein, 21 aa, Artificial Sequence
  Synthetic
MKITQEEVTHVANLSKLRFSE

SEQ ID NO: 42 - protein, 15 aa, Artificial Sequence
  Synthetic
HEKAVGDALDLSHAL

SEQ ID NO: 43 - protein, 18 aa, Artificial Sequence
  Synthetic
SIVTQVVESGFKGIFLVA

SEQ ID NO: 44 - protein, 40 aa, Artificial Sequence
  Synthetic
STKQHKKVILVGDGAVGSSYAFALVNQGIAQELGIIEIPQ

SEQ ID NO: 45 - protein, 44 aa, Artificial Sequence
  Synthetic
SPKKIYAAQYSDCADADLVVITAGAPQKPGETRLDLVGKNLAIN

SEQ ID NO: 46 - protein, 11 aa, Artificial Sequence
  Synthetic
VLDGTETVISG

SEQ ID NO: 47 - protein, 30 aa, Artificial Sequence
  Synthetic
YTGDQMILDGPHRGGDLRRARAGAANIVPN

SEQ ID NO: 48 - protein, 14 aa, Artificial Sequence
  Synthetic
ALRKVFEQESIDAA

SEQ ID NO: 49 - protein, 20 aa, Artificial Sequence
  Synthetic
VELLNLGKEVIIVDNLSNSS

SEQ ID NO: 50 - protein, 24 aa, Artificial Sequence
  Synthetic
LVLDRIEAITGIRPVFYELDVCDK

SEQ ID NO: 51 - protein, 26 aa, Artificial Sequence
  Synthetic
VMYYKNNIMSTLALVEVMSEFNVKKI

SEQ ID NO: 52 - protein, 30 aa, Artificial Sequence
  Synthetic
NPYGYTKVMLEQILKDVHVADSEWSIALLR

SEQ ID NO: 53 - protein, 15 aa, Artificial Sequence
  Synthetic
DGSFDIDPEIFELGI

SEQ ID NO: 54 - protein, 34 aa, Artificial Sequence
  Synthetic
MQFELIEPLNTLYKDEVRALGTELGMPDHIVWRQ

SEQ ID NO: 55 - protein, 16 aa, Artificial Sequence
  Synthetic
MTSAKEYIQSVFETVK

SEQ ID NO: 56 - protein, 14 aa, Artificial Sequence
  Synthetic
QGVICVSEGANMPS

SEQ ID NO: 57 - protein, 12 aa, Artificial Sequence
  Synthetic
LDAIKVYKENGI

SEQ ID NO: 58 - protein, 43 aa, Artificial Sequence
  Synthetic
RNGHEAEFLQAVEEFFNTLEPVFEKHPEYIEENILARITEPER

SEQ ID NO: 59 - protein, 47 aa, Artificial Sequence
  Synthetic
MRFCQSFMTELQKHIGPSLDVPAGDIGVGGREIGYLYGQYKRLNQFD

SEQ ID NO: 60 - protein, 24 aa, Artificial Sequence
  Synthetic
GVLTGKPLGFGGSLIRPEATGYGL

SEQ ID NO: 61 - protein, 44 aa, Artificial Sequence
  Synthetic
YYTEEMLKANGNSFAGKKVVISGSGNVAQYALQKATELGATVIS

SEQ ID NO: 62 - protein, 42 aa, Artificial Sequence
  Synthetic
SDSNGYVIDENGIDFDLLVDVKEKRRARLTEYAAEKATATYH

SEQ ID NO: 63 - protein, 26 aa, Artificial Sequence
  Synthetic
YAGNYDIALPCATQNEINGEAAKRLV

SEQ ID NO: 64 - protein, 34 aa, Artificial Sequence
  Synthetic
YGPAKAANAGGVAVSALEMSQNSLRLSWTREEVD

SEQ ID NO: 65 - protein, 46 aa, Artificial Sequence
  Synthetic
RLKDIMTNIFNTAKTTSETYGLDKDYLAGANIAAFENVANAMIAQG

SEQ ID NO: 66 - protein, 17 aa, Artificial Sequence
  Synthetic
NDFEAEVAATMAAALNN

SEQ ID NO: 67 - protein, 19 aa, Artificial Sequence
  Synthetic
LEDGQVLLVENTRYEDVDG

SEQ ID NO: 68 - protein, 38 aa, Artificial Sequence
  Synthetic
DKAGKSLAPVAADLAAKLGQDVVFPGVTRGAELEAAIN

SEQ ID NO: 69 - protein, 21 aa, Artificial Sequence
  Synthetic
KESKNDPELGKYWASLGDGIF

SEQ ID NO: 70 - protein, 11 aa, Artificial Sequence
  Synthetic
DAVAVEAEFAA

SEQ ID NO: 71 - protein, 19 aa, Artificial Sequence
  Synthetic
ETQADSIEEIVEVVEGDNA

SEQ ID NO: 72 - protein, 48 aa, Artificial Sequence
  Synthetic
RLEKFLGGIEDMPRIPDVMYVVDPHKEQIAVKEAKKLGIPVVAMVDTN

SEQ ID NO: 73 - protein, 36 aa, Artificial Sequence
  Synthetic
DPDDIDVIIPANDDAIRAVKLITAKLADAIIEGRQG

SEQ ID NO: 74 - protein, 11 aa, Artificial Sequence
  Synthetic
IRRNEELANSG

SEQ ID NO: 75 - protein, 18 aa, Artificial Sequence
  Synthetic
LSRKDDEGQDGPIVDYIL

SEQ ID NO: 76 - protein, 18 aa, Artificial Sequence
  Synthetic
LDVTAKYQQAVRDIVALA

SEQ ID NO: 77 - protein, 14 aa, Artificial Sequence
  Synthetic
PGTDATIQALLPHL

SEQ ID NO: 78 - protein, 10 aa, Artificial Sequence
  Synthetic
QDGPIVDYIL

SEQ ID NO: 79 - protein, 13 aa, Artificial Sequence
  Synthetic
VHASKVLPKPAAF

SEQ ID NO: 80 - protein, 9 aa, Artificial Sequence
  Synthetic
GDKAELIEK

SEQ ID NO: 81 - protein, 18 aa, Artificial Sequence
  Synthetic
AQLRVASKENNWNLPFAD

SEQ ID NO: 82 - protein, 20 aa, Artificial Sequence
  Synthetic
VPVPTFSAAITYFDSYRSAD

SEQ ID NO: 83 - protein, 12 aa, Artificial Sequence
  Synthetic
RKDKEGTFHYSW

SEQ ID NO: 84 - protein, 37 aa, Artificial Sequence
  Synthetic
ESRGYTVAIYNRSKEKTEDVIACHPEKNFVPSYDVES

SEQ ID NO: 85 - protein, 26 aa, Artificial Sequence
  Synthetic
DVLEEISAKAPEDGKPCVTYIGPDGA

SEQ ID NO: 86 - protein, 23 aa, Artificial Sequence
  Synthetic
AESYDLMQHLLGLSAEDMAEIFT

SEQ ID NO: 87 - protein, 9 aa, Artificial Sequence
  Synthetic
MNAIQESFT

SEQ ID NO: 88 - protein, 29 aa, Artificial Sequence
  Synthetic
KLFANYEANVKYQAIENAASHNGIFAALE

SEQ ID NO: 89 - protein, 50 aa, Artificial Sequence
  Synthetic
KWKVENSWGDKVGTDGYFVASDAWMDEYTYQIVVRKELLTAEEQAAYGAE

SEQ ID NO: 90 - protein, 11 aa, Artificial Sequence
  Synthetic
SFKYYVIEVNP

SEQ ID NO: 91 - protein, 11 aa, Artificial Sequence
  Synthetic
GVHHNEIPELA

SEQ ID NO: 92 - protein, 31 aa, Artificial Sequence
  Synthetic
RTDIQKIMVIGSGPIIIGQAAEFDYAGTQAC

SEQ ID NO: 93 - protein, 46 aa, Artificial Sequence
  Synthetic
SLKEEGYEVVLVNSNPATIMTDKEIADKVYIEPITLEFVTRILRKE

SEQ ID NO: 94 - protein, 29 aa, Artificial Sequence
  Synthetic
PDALLPTLGGQTGLNMAMELSKNGILDEL

SEQ ID NO: 95 - protein, 35 aa, Artificial Sequence
  Synthetic
SAIDQAEDRDLFKQLMEELEQPIPESEIVNTVEEA

SEQ ID NO: 96 - protein, 36 aa, Artificial Sequence
  Synthetic
QTMSDYENQMLRDASLSIIRALKIEGGCNVQLALDP

SEQ ID NO: 97 - protein, 21 aa, Artificial Sequence
  Synthetic
TAKLNGFSDRKIAELWGTTDD

SEQ ID NO: 98 - protein, 42 aa, Artificial Sequence
  Synthetic
VRQLRLENKIVPVYKMVDTCAAEFDSETPYFYSTYGWENESI

SEQ ID NO: 99 - protein, 41 aa, Artificial Sequence
  Synthetic
SDKESVLVLGSGPIRIGQGVEFDYATVHSVKAIQAAGYEAI

SEQ ID NO: 100 - protein, 27 aa, Artificial Sequence
  Synthetic
VLIPGYVIEVNPRASRTVPFLSKVTNI

SEQ ID NO: 101 - protein, 17 aa, Artificial Sequence
  Synthetic
IIENLPKVMDKYGISSL

SEQ ID NO: 102 - protein, 9 aa, Artificial Sequence
  Synthetic
EELRQEVKE

SEQ ID NO: 103 - protein, 38 aa, Artificial Sequence
  Synthetic
MTTNRLQVSLPGLDLKNPIIPASGCFGFGQEYAKYYDL

SEQ ID NO: 104 - protein, 28 aa, Artificial Sequence
  Synthetic
EAALEMYLAGASAIGVGTANFTNPYACP

SEQ ID NO: 105 - protein, 14 aa, Artificial Sequence
  Synthetic
AAQMDGAILVVAST

SEQ ID NO: 106 - protein, 19 aa, Artificial Sequence
  Synthetic
QERILEDINLQVTSGEVVS

SEQ ID NO: 107 - protein, 33 aa, Artificial Sequence
  Synthetic
IVSEIKLDWSEDEDKEVQKIAYKRQILAELGLD

SEQ ID NO: 108 - protein, 14 aa, Artificial Sequence
  Synthetic
LLKEFQELETFADF

SEQ ID NO: 109 - protein, 12 aa, Artificial Sequence
  Synthetic
AKLAEFELAGKP

SEQ ID NO: 110 - protein, 10 aa, Artificial Sequence
  Synthetic
LSGVPEAKDK

SEQ ID NO: 111 - protein, 9 aa, Artificial Sequence
  Synthetic
YKEWGMPAH

SEQ ID NO: 112 - protein, 43 aa, Artificial Sequence
  Synthetic
FLPFGVSPDFMDARINVLWASAPSTILPDTTYYAEEHPQREEL

SEQ ID NO: 113 - protein, 35 aa, Artificial Sequence
  Synthetic
TLWKESSANLLKAYDFSDEEIEDLLEKRLELDRRV

SEQ ID NO: 114 - protein, 27 aa, Artificial Sequence
  Synthetic
WSLLKATLILSVVNLSTSYLTEDIRVL

SEQ ID NO: 115 - protein, 13 aa, Artificial Sequence
  Synthetic
YQRVYRYLDKRVD

SEQ ID NO: 116 - protein, 18 aa, Artificial Sequence
  Synthetic
LKDSRTAKYHKRLQIVLF

SEQ ID NO: 117 - protein, 37 aa, Artificial Sequence
  Synthetic
ILDACPLVLDCRVDRIVEEDGICHIFAKILERLVAPE

SEQ ID NO: 118 - protein, 21 aa, Artificial Sequence
  Synthetic
LDEKGHFKNQLFAPTYFMGDG

SEQ ID NO: 119 - protein, 21 aa, Artificial Sequence
  Synthetic
MNIIEEIMTKLREDIRNIAII

SEQ ID NO: 120 - protein, 35 aa, Artificial Sequence
  Synthetic
VDELLKQSETLDARTELAERAMDSNDIEKERGITI

SEQ ID NO: 121 - protein, 44 aa, Artificial Sequence
  Synthetic
EVVDEVLELFIELGADDDQLDFPVVYASAINGTSSLSDDPADQE

SEQ ID NO: 122 - protein, 508 aa, Artificial Sequence
  Synthetic
MNAIQESFTAAANDFEAEVAATMAAALNNAAALEDGQVLLVENTRYEDVDGAAADKAGKSLAPVAADLAAKLGQDVVFPGVTRGAELEAAINAAAKESKNDPELGKYWASLGDGIFAAADAVAVEAEFAAAETQADSIEEIVEVVEGDNAAARLEKFLGGIEDMPRIPDVMYVVDPHKEQIAVKEAKKLGIPVVAMVDTNAAADPDDIDVIIPANDDAIRAVKLITAKLADAIIEGRQGAAAIRRNEELANSGAAALSRKDDEGQDGPIVDYILAAALDVTAKYQQAVRDIVALAAAAPGTDATIQALLPHLAAAQDGPIVDYILAAAVHASKVLPKPAAFAAAGDKAELIEKAAAQLRVASKENNWNLPFADAAAVPVPTAAASAAITYFDSYRSADAAARKDKEGTFHYSWAAAESRGYTVAIYNRSKEKTEAAAVIACHPEKNFVPSYDVESAAADVLEEISAKAPEDGKPCVTYIGPDGAAAESYDLMQHLLGLSAEDMAEIFT

SEQ ID NO: 123 - DNA, 1544 bp, Artificial Sequence
  Synthetic
catatgaatgctattcaagaatcttttactgctgctgctaatgattttgaagctgaagttgctgcaactatggctgcagcactgaacaacgccgcagcactggaagatggtcaggtactgctggtcgaaaacacccgctacgaagacgtcgacggtgctgcagcagataaagcaggcaaatccctggcgccagtagcagctgacctggcagcaaaactgggccaggacgtggtattcccaggtgtaacccgtggtgcagaactggaagcagcgatcaacgcagcggcgaaagaatccaaaaacgatccggaactgggtaaatactgggcctccctgggtgacggtattttcgcagctgcagacgcagtagctgttgaagcagaattcgctgcagcggaaacccaggctgacagcatcgaggaaattgtggaagtcgtagaaggcgacaacgcagctgcacgtctggagaaattcctgggtggcatcgaggatatgccacgcatcccggatgtcatgtacgttgtggacccgcacaaagagcagattgcggtcaaagaggccaaaaaactgggtatcccggtggtggcaatggtagataccaacgcagctgcggatccagacgacatcgacgtcatcattccggcgaacgacgacgctatccgtgcagtcaaactgatcacggcgaaactggcagacgccatcatcgaaggccgtcagggtgcagcagcgattcgtcgtaacgaggaactggctaacagcggtgctgctgctctgtctcgtaaagacgacgaaggtcaggacggtccgatcgtcgactatattctggcagcagcactggacgtcaccgcaaaataccagcaggctgttcgtgacattgttgcactggctgcggcagctccaggtactgatgcgaccattcaggcactgctgccacatctggctgctgcacaggatggtccgatcgtcgattacattctggctgcagcggttcacgcaagcaaagtcctgccgaaaccggctgcttttgcagctgctggcgacaaagctgaactgatcgagaaagctgctgcacagctgcgcgtcgcgtctaaagagaacaactggaacctgccgttcgctgacgcagctgcagttccagttccaactgccgcggctagcgctgcgattacgtatttcgactcctatcgttccgcagatgcagcagcacgcaaagataaagagggcacgttccactattcctgggcagcggctgaatctcgtggctatacggtcgctatctacaaccgctccaaagagaaaacggaggcagcagcagttatcgcgtgccacccggaaaaaaacttcgtgccgtcttacgacgtcgaaagcgcagctgcagatgtgctggaggagatctctgctaaagctccggaggacggcaaaccgtgtgtgacttacatcggtccggatggtgcagctgctgaaagctacgacctgatgcagcatctgctgggtctgagcgcagaggatatggccgagatcttcacgtaataagcttgctgagc

SEQ ID NO: 124 - protein, 492 aa, Artificial Sequence
  Synthetic
MKEKAMEVPAIKAAAKLFANYEANVKYQAIENAASHNGIFAALEAAAKWKVENSWGDKVGTDGYFVASDAWMDEYTYQIVVRKELLTAEEQAAYGAEAAASFKYYVIEVNPAAAGVHHNEIPELAAAARTDIQKIMVIGSGPIIIGQAAEFDYAGTQACAAASLKEEGYEVVLVNSNPATIMTDKEIADKVYIEPITLEFVTRILRKEAAADALLPPTLGGQTGLNMAMELSKNGILDELAAASAIDQAEDRDLFKQLMEELEQPIPESEIVNTVEEAAAAQTMSDYENQMLRDASLSIIRALKIEGGCNVQLALDPAAATAKLNGFSDRKIAELWGTTDDAAAVRQLRLENKIVPVYKMVDTCAAEFDSETPYFYSTYGWENESIAAASDKESVLVLGSGPIRIGQGVEFDYATVHSVKAIQAAGYEAIAAAVLIPGYVIEVNPRASRTVPFLSKVTNIAAAIIENLPKVMDKYGISSLAAAEELRQEVKE

SEQ ID NO: 125 - DNA, 1496 bp, Artificial Sequence
  Synthetic
catatgaaagaaaaagctatggaagttccagctattaaagctgctgcaaaactgtttgctaattatgaagccaacgtcaaataccaggcgattgaaaacgctgcgtcccacaacggcatctttgcggcgctggaggccgctgcgaaatggaaagttgagaactcttggggtgacaaagtgggtactgatggctacttcgtagccagcgatgcttggatggacgagtatacctaccagattgtagtgcgcaaagaactgctgaccgctgaggaacaggcagcctatggtgcggaggcggcagcatccttcaaatactatgttatcgaagtgaacccggcggctgcaggtgtgcatcacaacgagatcccggaactggcggcggcggctcgtaccgatatccagaaaatcatggtcatcggtagcggtccgatcatcattggtcaggcggcagaatttgactacgcaggtacccaggcatgtgctgctgcctctctgaaagaagaaggctacgaggtagttctggtaaactccaacccggcaactatcatgaccgacaaagagatcgcggacaaagtgtacattgaaccgatcaccctggaattcgttacgcgtattctgcgtaaagaagctgcggctgatgcgctgctgccaccgactctgggtggccagaccggtctgaacatggctatggaactgagcaaaaacggcatcctggatgaactggcggccgctagcgctatcgatcaggcggaagatcgcgatctgttcaaacagctgatggaggaactggagcagccgatcccggaatccgaaattgtaaacactgtggaagaagcggcggcggctcagaccatgtccgactacgaaaaccagatgctgcgtgacgcctctctgtctatcattcgtgcgctgaaaatcgaaggtggctgcaacgttcagctggctctggatccggcagcagcaaccgcgaaactgaacggcttctccgaccgcaaaatcgctgaactgtggggtaccactgatgatgctgctgctgtacgccagctgcgtctggaaaacaaaatcgttccggtttacaaaatggtggacacctgcgcagccgaattcgactctgaaactccgtacttctattctacgtacggttgggaaaacgagtctatcgcagccgcctctgacaaagaatctgtactggttctgggcagcggcccgattcgcatcggtcagggtgttgaattcgactacgcgaccgtccactccgtgaaagcaattcaggcagcaggctatgaagccatcgcggcagccgtgctgattccaggctatgttatcgaagttaacccgcgtgcctctcgtaccgttccgttcctgtctaaagtgacgaacatcgccgcagcgatcattgaaaacctgccgaaagttatggacaaatacggcatcagctctctggcagcggcggaggaactgcgtcaggaagtcaaagagtaataagcttgctgagc

SEQ ID NO: 126 - protein, 513 aa, Artificial Sequence
  Synthetic
MKEKAMEVPAIKAAAVAYLVETEGASAGVMIAAAYYVDTITDVVRAEIGIDAAASHNPALDNGIKFFGGDGFKLDDEKEAEIEALLDAEEDTLPAAAIGLAFDGDSDRAAAVDENGDIVDGDKIMYIIGKYLSAAATGKSLSELAAEVTIYPQKLVNIRVENAAAIIEKMEEEMAGNGRILVRPSGTEPLLRVMAEAPTAAARDRLFKNVPEAAADNYYIKVPAILDAAAETAAFATTLSKIVDMVELLGEVDTTGVAPTTTMADRKTVLRPDVAEEGAAAMKITQEEVTHVANLSKLRFSEAAAHEKAVGDALDLSHALAAASIVTQVVESGFKGIFLVAAASTKQHKKVILVGDGAVGSSYAFALVNQGIAQELGIEIPQAAASPKKIYAAQYSDCADADLVVITAGAPQKPGETRLDLVGKNLAINAAAVLDGTETVISGAAAYTGDQMILDGPHRGGDLRRARAGAANIVPNAAALRKVFEQESIDAAAVELLNLGKEVIIVDNLSNSS

SEQ ID NO: 127 - DNA, 1561 bp, Artificial Sequence
  Synthetic
catatgaaagaaaaagctatggaagttccagctattaaagctgctgctgttgcttacctggttgaaaccgaaggcgcttctgctggtgtgatgattgcggcagcgtactatgtagacactatcaccgacgttgtccgtgcggagattggcattgatgcagctgcttctcacaacccagcgctggataacggcatcaaatttttcggcggtgacggcttcaaactggatgacgagaaagaagccgagattgaagctctgctggacgcagaagaggatacgctgccggcagccgcaatcggtctggctttcgacggcgattctgatcgcgcagccgcagtggatgagaacggtgatatcgtagacggcgacaaaattatgtacatcatcggcaaatacctgagcgctgcagcgaccggtaaatccctgtctgaactggccgctgaagtcacgatttacccgcagaaactggttaacatccgcgtagaaaacgccgccgcgattatcgaaaaaatggaggaagagatggcgggtaacggtcgtatcctggttcgcccgtctggcactgaaccgctgctgcgcgtgatggctgaagcgccaactgctgcggctcgtgaccgtctgttcaaaaacgttccggaagcggcagccgacaactattacatcaaagtgccagcgattctggatgctgccgctgaaaccgctgccttcgcgaccacgctgtccaaaatcgtcgacatggtcgaactgctgggtgaagttgataccaccggtgtagccccgaccacgactatggcggaccgcaaaactgttctgcgtccggacgttgctgaagagggtgctgcagccatgaaaatcacccaggaagaggtgactcatgtagcgaacctgtctaaactgcgtttcagcgaagcggctgcacacgagaaagcggttggtgacgcactggacctgtctcatgccctggctgctgcgagcatcgtaacccaggtagttgaatccggttttaaaggtatctttctggtggcggcagcctctactaaacagcacaaaaaagtgatcctggtgggtgacggtgctgttggctcctcttatgcgttcgcactggtgaaccagggtattgcgcaggaactgggtatcgaaatcccacaggctgcagcttccccgaaaaaaatctacgctgcccagtactctgattgcgcagacgcggatctggtagttatcactgcaggtgcaccgcagaaaccaggtgaaacccgcctggacctggtgggcaaaaacctggcgattaacgcagctgcagtcctggatggcaccgaaaccgttatctctggtgcagctgcctataccggtgatcagatgatcctggatggcccgcaccgtggtggtgatctgcgtcgtgctcgtgcaggcgctgcaaacattgttccgaacgctgcagcgctgcgtaaagtattcgaacaggagtccatcgacgctgcagctgttgaactgctgaacctgggcaaagaagtgatcatcgtggataacctgagcaacagcagctaataaaagcttgctgagc

SEQ ID NO: 128 - protein, 522 aa, Artificial Sequence
  Synthetic
MTSAKEYIQSVFETVKAAALVLDRIEAITGIRPVFYELDVCDKAAAVMYYKNNIMSTLALVEVMSEFNVKKIAAANPYGYTKVMLEQILKDVHVADSEWSIALLRAAADGSFDIDPEIFELGIAAAMQFELIEPLNTLYKDEVRALGTELGMPDHIVWRQAAAQGVICVSEGANMPSAAALDAIKVYKENGIAAARNGHEAEFLQAVEEFFNTLEPVFEKHPEYIEENILARITEPERAAAMRFCQSFMTELQKHIGPSLDVPAGDIGVGGREIGYLYGQYKRLNQFDAAAGVLTGKPLGFGGSLIRPEATGYGLAAAYYTEEMLKANGNSFAGKKVVISGSGNVAQYALQKATELGATVISAAASDSNGYVIDENGIDFDLLVDVKEKRRARLTEYAAEKATATYHAAAYAGNYDIALPCATQNEINGEAAKRLVAAAYGPAKAANAGGVAVSALEMSQNSLRLSWTREEVDAAARLKDIMTNIFNTAKTTSETYGLDKDYLAGANIAAFENVANAMIAQG

SEQ ID NO: 129 - DNA, 1588 bp, Artificial Sequence
  Synthetic
catatgacttctgctaaagaatatattcagtctgtttttgaaactgttaaagcagctgctctggtgctggatcgtatcgaagctatcacgggcattcgcccggtgttctatgaactggatgtttgcgacaaagcggcagcagtgatgtactataaaaacaacatcatgtccaccctggcgctggtcgaagtgatgtctgaattcaacgttaaaaaaattgctgctgcgaacccgtacggctacacgaaagtgatgctggaacagatcctgaaagatgtgcacgttgcggattccgaatggtctattgcactgctgcgtgcggctgcggacggcagcttcgacattgacccggaaattttcgaactgggtatcgcggcagcaatgcagttcgaactgatcgaaccgctgaacaccctgtataaagatgaagttcgtgcactgggcaccgagctgggcatgccggaccacatcgtttggcgccaggccgccgctcagggtgttatctgtgttagcgagggtgcaaacatgccatccgcggccgcactggacgcaatcaaagtatataaagagaacggcatcgcggctgcacgtaacggccacgaggcggaattcctgcaggcggtagaagaatttttcaacaccctggaaccggtgtttgagaaacacccggaatacattgaagaaaacattctggcccgcatcaccgaaccggaacgcgcggctgcgatgcgtttctgccagtctttcatgactgaactgcagaaacatatcggtccgtccctggacgttccagcgggtgatattggcgtaggtggtcgtgaaattggctacctgtacggccagtacaaacgtctgaaccagtttgacgcggctgctggtgtgctgacgggcaaaccgctgggtttcggtggtagcctgatccgtccagaggctactggttacggtctggcggctgcctactacaccgaggaaatgctgaaagcaaacggcaacagcttcgctggcaaaaaagttgtaatctctggtagcggtaacgtcgcacagtacgctctgcagaaagctaccgagctgggtgctaccgtaatctccgctgcagcctccgattctaacggttatgtgatcgacgaaaacggtatcgactttgacctgctggttgacgtaaaagaaaaacgtcgtgcgcgcctgaccgaatatgcggccgagaaagcaactgctacctatcatgccgcggcctacgcgggcaactacgacatcgcactgccatgtgcgactcagaacgaaatcaacggcgaagcggcgaaacgtctggtcgcggctgcgtacggtccagcaaaagctgctaacgccggtggtgttgccgtatctgccctggaaatgtctcagaactctctgcgcctgagctggacccgtgaagaggtcgatgcggccgcacgtctgaaagatatcatgaccaacatcttcaacaccgcgaaaactacgtctgagacctacggtctggataaagattacctggctggcgcaaacatcgcggcattcgaaaacgttgctaacgccatgattgcacagggctaataaaagcttgctgagc

SEQ ID NO: 130 - protein, 522 aa, Artificial Sequence
  Synthetic
MTTNRLQVSLPGLDLKNPIIPASGCFGFGQEYAKYYDLAAAEAALEMYLAGASAIGVGTANFTNPYACPAAAQMDGAILVVASTAAAQERILEDINLQVTSGEVVSAAAIVSEIKLDWSEDEDKEVQKIAYKRQILAELGLDAAALLKEFQELETFADFAAAKLAEFELAGKPAAALSGVPEAKDKAAAYKEWGMPAHAAAFLPFGVSPDFMDARINVLWASAPSTILPDTTYYAEEHPQREELAAATLWKESSANLLKAYDFSDEEIEDLLEKRLELDRRVAAAWSLLKATLILSVVNLSTSYLTEDIRVLAAAYQRVYRYLDKRVDAAALKDSRTAKYHKRLQIVLFAAAILDACPLVLDCRVDRIVEEDGICHIFAKILERLVAPEAAALDEKGHFKNQLFAPTYFMGDGAAAMNIIEEIMTKLREDIRNIAIIAAAVDELLKQSETLDARTELAERAMDSNDIEKERGITIAAAEVVDEVLELFIELGADDDQLDFPVVYASAINGTSSLSDDPADQE

SEQ ID NO: 131 - DNA, 1588 bp, Artificial Sequence
  Synthetic
catatgactactaaccgtctgcaagtatctctgccaggtctggatctgaaaaacccgattattccggcatccggctgctttggctttggtcaggagtacgccaaatactatgacctggctgcagccgaagcggcactggaaatgtatctggctggtgctagcgcaattggcgtaggtacggctaactttacgaacccgtacgcctgtccagctgcagcccagatggatggcgcaatcctggtagtggcttccactgctgccgctcaggaacgcattctggaagacatcaacctgcaggtgacttctggcgaggtagtgtctgctgcagcgatcgtctctgagatcaaactggattggagcgaggacgaggacaaagaagtgcagaaaatcgcatacaaacgtcagatcctggctgaactgggtctggatgctgcagcgctgctgaaagagttccaggagctggaaacctttgcggactttgctgcagccaaactggcagaattcgaactggctggtaaaccggcagcggcactgtctggcgtgccagaagcaaaagacaaagcggcagcttacaaagagtggggtatgccggcacacgcagccgcttttctgccgtttggtgtgtctccggacttcatggacgcacgtatcaacgttctgtgggcctctgctccatctactatcctgccggataccacgtactatgcggaggaacacccacagcgtgaggaactggcagctgcaaccctgtggaaagaatccagcgcaaacctgctgaaagcgtacgatttcagcgacgaagagatcgaggatctgctggagaaacgcctggaactggaccgtcgcgtagctgcagcttggagcctgctgaaagccactctgatcctgagcgtagtcaacctgagcacgagctatctgacggaagacattcgcgttctggcagctgcctatcagcgtgtataccgctatctggacaaacgcgttgacgcagctgccctgaaagattctcgcactgcgaaataccacaaacgtctgcagatcgtcctgttcgctgccgcaattctggacgcttgtccgctggtcctggattgtcgtgtagaccgtatcgtagaggaagacggtatctgtcacatcttcgccaaaattctggaacgcctggtagctccggaagctgcagctctggacgagaaaggtcacttcaaaaaccagctgttcgcaccaacctacttcatgggtgacggtgcagccgcaatgaacattatcgaggagatcatgacgaaactgcgtgaggacatccgcaacattgcaatcatcgctgcagcggtagacgaactgctgaaacagagcgaaactctggacgctcgtactgaactggcagagcgtgcaatggactccaacgatatcgagaaagagcgtggtatcactatcgctgcggcagaagttgtagacgaggtcctggagctgtttatcgagctgggtgcagacgatgaccagctggatttcccggtggtctatgcttccgctatcaacggcacctcctctctgagcgatgacccagctgatcaggagtaataaaagcttgctgagc

SEQ ID NO: 132 - protein, 15 aa, Artificial Sequence
  Synthetic
MAAGAAEAAVAAVEE

SEQ ID NO: 133 - protein, 13 aa, Artificial Sequence
  Synthetic
DNVLDHLTGRSCQ

(19) United States
(12) Patent Application Publication
(10) Pub. No.: US 2012/0100172 A1
(43) Pub. Date: Apr. 26, 2012

(54) IMMUNOGENIC STREPTOCOCCUS PNEUMONIAE PEPTIDES AND PEPTIDE-MULTIMERS

(76) Inventors: Michael Tal, Kefar Bilu (IL); Maxim Portnoi, Beer Sheva (IL); Ron Dagan, Omer (IL); Yaffa Mizrachi-Nebenzahl, Beer Sheva (IL)

(21) Appl. No.: 13/379,330

(22) PCT Filed: Jun. 3, 2010
(86) PCT No.: PCT/IL10/00439

§ 371 Date: Dec. 19, 2011

(30) Foreign Application Priority Data
Jun. 25, 2009 (US) 61220222

(51) Int. Cl. A61K 39/09; C12N 15/63; A61P 37/04; C07K 7/08; C07K 7/06; C07H 21/04; C12N 1/21; C07K 14/315

(52) U.S. Cl. 424/190.1; 435/252.33; 435/320.1; 530/322; 530/324; 530/325; 530/326; 530/327; 530/328; 530/350; 536/23.7

(57) ABSTRACT

The present invention relates to immunogenic peptides, including variants and analogs derived from Streptococcus pneumoniae (S. pneumoniae) proteins, to peptide-multimers, conjugates and fusion proteins that include such peptides, and to vaccines that include such immunogenic entities. In particular, the present invention relates to the use of such vaccines for eliciting protective immunity to S. pneumoniae.

### FIELD OF THE INVENTION

The present invention relates to immunogenic peptides derived from Streptococcus pneumoniae (S. pneumoniae) cell wall or cell membrane proteins and to their use in protection against infection with the bacteria. In particular, the present invention relates to immunogenic peptides derived from cell wall or cell membrane proteins of S. pneumoniae which exhibit age-dependent immunity against the bacteria and multimers produced from these peptides.

### BACKGROUND OF THE INVENTION

Streptococcus pneumoniae belongs to the commensal flora of the human respiratory tract, but can also cause invasive infections such as meningitis and sepsis. Mortality due to pneumococcal infection remains high all over the world, augmented by a wide-spread antibiotic resistance in many pneumococcal strains. The current polysaccharide based vaccines (including polysaccharide conjugates), elicit a strain specific protection in children and the elderly, who are the main targets for pneumococcal infections. However the available vaccines either do not elicit long lasting protection or are limited in strain coverage. Development of new preventive and therapeutic interventions is hampered due to the incomplete understanding of pneumococcal pathogenesis.

Most children in the developing world become nasopharyngeal carriers of Streptococcus pneumoniae. Many develop pneumococcal disease that can be invasive (such as bacteremia, sepsis or meningitis), or mucosal infections (such as pneumonia and otitis media). S. pneumoniae is the leading cause of non-epidemic childhood meningitis in Africa and other regions of the developing world. Approximately, one to two million children die from pneumococcal inflicted diseases each year. Specifically, when considering deaths of children under five years of age worldwide, about 20% is from pneumococcal pneumonia. These high morbidity and mortality rates and the persistent emergence of antibiotic resistant strains of S. pneumoniae heighten the need to develop an effective means of prevention, such as vaccination. The optimal anti-pneumococcal vaccine should be safe, efficacious, wide-spectrum (covering most pneumococcal strains) and affordable (cheap and available in large quantities).

The mucosal epithelial surfaces with their tight junctions constitute the first line of defense that prevents the entry of pathogens and their products. S. pneumoniae adhere to the nasopharyngeal mucosal cells (Tuomanen E. 1999 Curr. Opin. Microbiol., 2:35-9) causing carriage without an overt inflammatory response. For clinical disease to occur, S. pneumoniae have to spread from the nasopharynx into the middle ear or the lungs or cross the mucosal epithelial cell layer and be deposited basally within the submucosa (Ring et al., J. Clin. Invest. 1998, 102:347-60). Molecules involved in adhesion, spread and invasion of S. pneumoniae, include capsular polysaccharides, cell-wall peptidoglycan and surface proteins (Jedrzejas M J. Microbiol. Mol. Biol. Rev. 2001, 65, 187-207).

The search for wide range anti-pneumococcal vaccine is ongoing. Indeed, introduction of pneumococcal 7-valent polysaccharide conjugate vaccine reduced significantly the rates of invasive diseases in infants and restricted significantly the rates of invasive diseases in the non vaccinated members of the community (Kyaw et al., N. Engl. J. Med. 2006, 354, 1455-63). However, carriage and diseases resulting from strains not included in the vaccine are on the rise (Musher D M., N. Engl. J. Med. 2006, 354, 1522-4, Huang et al., Pediatrics 2005, 116, e408-13). Vaccination with multivalent polysaccharide conjugate vaccines has been shown to be associated with serotype replacement, whereby non-vaccine serotype strains have elevated levels of carriage in populations with reduced incidence of vaccine serotype strains, which means that the effectiveness of conjugate vaccines may diminish over time.

The existing pneumococcal polysaccharide and polysaccharide-conjugated vaccines protect against a narrow but significant group of pneumococcal serotypes, vaccinated subjects remaining susceptible to strains not covered by the vaccines. The current pneumococcal conjugate vaccines generally have lower coverage against pneumococcal strains causing disease in the developing world compared to developed countries. In the developed world, Nasopharyngeal carriage of Streptococcus pneumoniae shortly before vaccination with a pneumococcal conjugate vaccine causes serotype-specific hyporesponsiveness in early infancy (Dagan R et al. J. Infec. Dis. 2010; 201:1570-1579). In addition to limitations of coverage, conjugate vaccines are complex to produce and expensive, resulting in restricted quantities and are beyond the budget of many poor countries.

It has been observed that in infants the antibody response to S. pneumoniae proteins increases with age and correlates negatively with morbidity (Lifshitz et al. Clin. Exp. Immunol. 2002, 127, 344-53). To identify these proteins a longitudinal series of children's sera from healthy children, exposed to bacterial infections, was collected and utilized to survey which S. pneumoniae cell wall associated proteins exhibit age-dependent antigenicity together with biochemical and proteomic studies, (Ling et al., Clin Exp Immunol 2004, 138, 290-8).

WO 2003/082183 to one of the inventors of the present application discloses a defined group of immunogenic cell wall and cell membrane S. pneumoniae proteins for use as vaccines against said bacteria. It was found that such vaccine compositions are effective against a wide range of different S. pneumonias serotypes, and in all age groups, including those age groups which do not produce anti-S. pneumonias antibodies following inoculation with polysaccharide-based vaccines.

Exposure to the intact bacteria in infants is insufficient to elicit an immune response. The enhancement, with age, of antibody responses against pneumococcal surface proteins, is denoted “age dependency” and the identified proteins are denoted “age dependent proteins”).

International Patent Application Publication No. WO 2002/077021, assigned to Chiron S.P.A., discloses the sequence of about 2,500 S. pneumoniae genes, including some of the bacteria intact genes, and their corresponding amino acid sequences from S. pneumoniae type 4 strain that were identified in silico. The use of a subset of 432 of those sequences as antigens for immunization is also suggested although no guidance in selecting useful proteins as antigens in the production of vaccines are provided.

Multi-epitope vaccines against influenza virus are disclosed in WO 2009/016639. Multiepitope DNA vaccines are discussed in Subbramanian et al. (J. Virol. 2003, 77, 10113-10118). Multivalent minigene vaccines containing B-cell, CTL and Th epitopes from several pathogens are described in Ling-Ling and Whitton (J. Virol 1997, 71 2292-2302).

There is an unmet need to provide short peptide epitopes, effective at eliciting immunity against a wide range of different S. pneumoniae serotypes and having minimal homology to human proteins. These epitopes would be useful in improved S. pneumoniae peptide-based vaccines which can induce long-lasting immunological responses, and in all age groups, including young children, immunocompromised subjects and elderly people.

### SUMMARY OF THE INVENTION

The present invention provides immunogenic peptides, peptide multimers and vaccines against S. pneumoniae. The peptides of the present invention are derived from S. pneumoniae “age-dependent” proteins namely, from proteins associated with an age-dependent immune response.

According to the present invention, isolation of antigenic epitopes from the bacteria cell wall proteins enables effective presentation of the antigenic determinants and increases their immunogenic potential against the bacteria. Multimeric constructs comprising plurality of peptide epitopes increases the protection efficacy and range by enabling simultaneous exposure to several antigenic determinants in one composition of matter.

The peptides of the present invention, which may retain the “age-dependency” of the proteins from which they are derived, have reduced homology to human sequences compared to the intact protein, minimizing the risk of developing antibodies against the patient's own proteins. Furthermore, the peptides of the present invention have very high sequence identity to many different S. pneumoniae strains making them ideal for wide-spectrum vaccine against the bacterium.

According to the present invention immunogenic peptides can be produced recombinantly, as isolated peptides or peptide-multimers, or as part of a fusion protein, or can be synthesized by peptide synthesis or by linking several, identical and/or different synthetic peptide fragments from same or different S. pneumoniae proteins. Recombinant or synthetic production can be used, according to the present invention, to introduce specific mutations and/or variations in the peptide sequence for improving specific properties such as solubility and stability.

The production of a peptide reduces the protein load and more immunogenic epitopes will be present per microgram of product. Advantageously, it will also be easier to purify in a consistent manner and to characterize analytically, thereby better addressing regulatory requirements.

The peptides of the present invention can be used in vaccines against S. pneumoniae alone, in mixture with other immunogenic peptides, protein fragments or proteins, as part of a chimeric protein which may be used as an adjuvant, or mixed or formulated with an external adjuvant.

The peptides of the present invention may be also used in conjunction or after conjugation with at least one carbohydrate moiety, for example an S. pneumoniae polysaccharides. Combination vaccines and conjugate compositions according to the invention may include at least one peptide antigen derived from an age-dependent S. pneumoniae protein, and at least one carbohydrate moiety, e.g. S. pneumoniae antigenic polysaccharide moiety.

A peptide according to the invention shares less than 78% sequence identity with a contiguous sequence of seven or more amino acid residues of a human protein. Accordingly, a peptide of the invention may contain no more than 7 contiguous amino acid residues identical to a contiguous amino acid sequence of a human protein.

In one aspect, the present invention provides a synthetic or recombinant peptide, peptide multimer or peptide conjugate comprising a sequence of 9-50 amino acids derived from the sequence of an S. pneumoniae protein.

In certain embodiments, the present invention provides a synthetic or recombinant polypeptide (herein denoted “peptide-multimer”) comprising a plurality of S. Pneumoniae derived peptides each peptide having 9-50 amino acids derived from the sequence of S. pneumoniae cell wall or cell membrane protein associated with an age-dependent immune response, and variants and analogs thereof. The peptide-multimer may contain a plurality of repeats not necessarily adjacent, of a specific peptide, a plurality of different peptides, a plurality of repeats of a plurality of peptides, or a combination of any of those options. The peptide-multimer may comprise peptides from one or more S. pneumoniae derived age-dependent proteins.

According to some specific embodiments a peptide multimer according to the present invention is selected from the groups consisting of SEQ ID NOs: 122, 124, 126, 128 and 130.

A peptide-multimer according to some embodiments is produced as part of a fusion protein comprising a carrier sequence which may serve as an adjuvant.

According to one embodiment, the fusion protein comprises detoxified pneumolysin or a fragment thereof. According to another embodiment, the fusion protein comprises heat shock protein 60 (hsp60) or a fragment thereof.

According to a specific embodiment the present invention provides a peptide-multimer comprising multiple copies of plurality of different S. Pneumoniae derived peptides, providing multi diversity, high density vaccine. According to the present invention the peptide-multimer can be produced recombinantly, as an isolated polypeptide or as a fusion protein, or synthetically by linking a plurality of synthetic peptides, or can be mixed or formulated with an external adjuvant or with another antigenic moiety such as a carbohydrate moiety.

According to some embodiments the present invention provides a synthetic or recombinant peptide-multimer comprising multiple copies of a plurality of S. pneumoniae derived peptides arranged in an alternating sequential polymeric structure (X₁X₂X₃ . . . )ₙ or in a block copolymer structure (X₁)ₙ(X₂)ₙ(X₃)ₙ . . . (Xₘ)ₙ.

A synthetic or recombinant peptide-multimer according to the present invention is selected according to a specific embodiment from the group consisting of:

i. B(X₁ZX₂Z . . . Xₘ)ₙB; and
ii. B(X₁)ₙZ(X₂)ₙZ . . . (Xₘ)ₙB;
wherein B is an optional sequence of 1-4 amino acid residues; n is at each occurrence independently an integer of 2-8; m is an integer of 2-8; each of X₁, X₂ . . . Xₘ is an immunogenic S. pneumoniae derived peptide consisting of 9-50 amino acid residues; Z at each occurrence is a bond or a spacer of 1-4 amino acid residues; and wherein the maximal number of amino acid residues in the peptide-multimer is about 900.

According to certain embodiments the spacer Z is selected from the group consisting of: Ala, Ala-Ala, Ala-Ala-Ala, Gly, Gly-Gly, Gly-Gly-Gly, Pro and Lys.

According to some embodiments at least one amino acid of the spacer induces a specific conformation on a segment of the polypeptide (e.g. a proline residue).

According to yet other embodiments the spacer comprises a cleavable sequence.

According to one embodiment the cleavable spacer is cleaved by intracellular enzymes. According to a more specific embodiment the cleavable spacer comprises a protease specific cleavable sequence.

In additional embodiments, the present invention provides a synthetic or recombinant peptide of 9-50 amino acids derived from the sequence of an S. pneumoniae protein.

According to some embodiments the synthetic or recombinant peptide or polypeptide comprises at least one peptide sequence of 9-50 amino acids, derived from the sequence of an S. pneumoniae protein associated with an age-dependent immune response, wherein the peptide sequence of 9-50 amino acids is selected from the group consisting of:

| | SEQ ID |
|---|---|
| | NO: |
| AVAYLVETEGASAGVMI | 26 |
| MKEKAMEVPAIK | 27 |
| YYVDTITDVVRAEIGID | 28 |
| ASHNPALDNGIKFFGGDGFKLDDEKEAEIEALLDAEEDTLP | 29 |
| IGLAFDGDSDRLIAVDENGDIVDGDKIMYIIGKYLS | 30 |
| TGKSLSELAAEVTIYPQKLVNIRVEN | 31 |
| IIEKMEEEMAGNGRILVRPSGTEPLLRVMAEAPT | 32 |
| TAQWNNHRV | 33 |
| LDIPAIKGINPDTD | 34 |
| GSYHDVDSSETAFKIAASL | 35 |
| HKIGETHEGASQMDWMEQEQE | 36 |
| LMMKYLEGEEITNEELKAGIRKATINVEFFPVLC | 37 |
| RDRLFKNVPE | 38 |
| DNYYIKVPAILD | 39 |
| ETAAFATTLSKIVDMVELLGEVDTTGVAPTTTMADRKTVLRPDVAEEG | 40 |
| MKITQEEVTHVANLSKLRFSE | 41 |
| HEKAVGDALDLSHAL | 42 |
| SIVTQVVESGFKGIFLVA | 43 |
| STKQHKKVILVGDGAVGSSYAFALVNQGIAQELGIIEIPQ | 44 |
| SPKKIYAAQYSDCADADLVVITAGAPQKPGETRLDLVGKNLAIN | 45 |
| VLDGTETVISG | 46 |
| YTGDQMILDGPHRGGDLRRARAGAANIVPN | 47 |
| ALRKVFEQESIDAA | 48 |
| VELLNLGKEVIIVDNLSNSS | 49 |
| LVLDRIEAITGIRPVFYELDVCDK | 50 |
| VMYYKNNIMSTLALVEVMSEFNVKKI | 51 |
| NPYGYTKVMLEQILKDVHVADSEWSIALLR | 52 |
| DGSFDIDPEIFELGI | 53 |
| MQFELIEPLNTLYKDEVRALGTELGMPDHIVWRQ | 54 |
| MTSAKEYIQSVFETVK | 55 |
| QGVICVSEGANMPS | 56 |
| LDAIKVYKENGI | 57 |
| RNGHEAEFLQAVEEFFNTLEPVFEKHPEYIEENILARITEPER | 58 |
| MRFCQSFMTELQKHIGPSLDVPAGDIGVGGREIGYLYGQYKRLNQFD | 59 |
| GVLTGKPLGFGGSLIRPEATGYGL | 60 |
| YYTEEMLKANGNSFAGKKVVISGSGNVAQYALQKATELGATVIS | 61 |
| SDSNGYVIDENGIDFDLLVDVKEKRRARLTEYAAEKATATYH | 62 |
| YAGNYDIALPCATQNEINGEAAKRLV | 63 |
| YGPAKAANAGGVAVSALEMSQNSLRLSWTREEVD | 64 |
| RLKDIMTNIFNTAKTTSETYGLDKDYLAGANIAAFENVANAMIAQG | 65 |
| NDFEAEVAATMAAALNN | 66 |
| LEDGQVLLVENTRYEDVDG | 67 |
| DKAGKSLAPVAADLAAKLGQDVVFPGVTRGAELEAAIN | 68 |
| KESKNDPELGKYWASLGDGIF | 69 |
| DAVAVEAEFAA | 70 |
| ETQADSIEEIVEVVEGDNA | 71 |
| RLEKFLGGIEDMPRIPDVMYVVDPHKEQIAVKEAKKLGIPVVAMVDTN | 72 |
| DPDDIDVIIPANDDAIRAVKLITAKLADAIIEGRQG | 73 |
| IRRNEELANSG | 74 |
| LSRKDDEGQDGPIVDYIL | 75 |
| LDVTAKYQQAVRDIVALA | 76 |
| PGTDATIQALLPHL | 77 |
| QDGPIVDYIL | 78 |
| VHASKVLPKPAAF | 79 |
| GDKAELIEK | 80 |
| AQLRVASKENNWNLPFAD | 81 |
| VPVPTFSAAITYFDSYRSAD | 82 |
| RKDKEGTFHYSW | 83 |
| ESRGYTVAIYNRSKEKTEDVIACHPEKNFVPSYDVES | 84 |
| DVLEEISAKAPEDGKPCVTYIGPDGA | 85 |
| AESYDLMQHLLGLSAEDMAEIFT | 86 |
| MNAIQESFT | 87 |
| KLFANYEANVKYQAIENAASHNGIFAALE | 88 |
| KWKVENSWGDKVGTDGYFVASDAWMDEYTYQIVVRKELLTAEEQAAYGAE | 89 |
| SFKYYVIEVNP | 90 |
| GVHHNEIPELA | 91 |
| RTDIQKIMVIGSGPIIIGQAAEFDYAGTQAC | 92 |
| SLKEEGYEVVLVNSNPATIMTDKEIADKVYIEPITLEFVTRILRKE | 93 |
| PDALLPTLGGQTGLNMAMELSKNGILDEL | 94 |
| SAIDQAEDRDLFKQLMEELEQPIPESEIVNTVEEA | 95 |
| QTMSDYENQMLRDASLSIIRALKIEGGCNVQLALDP | 96 |
| TAKLNGFSDRKIAELWGTTDD | 97 |
| VRQLRLENKIVPVYKMVDTCAAEFDSETPYFYSTYGWENESI | 98 |
| SDKESVLVLGSGPIRIGQGVEFDYATVHSVKAIQAAGYEAI | 99 |
| VLIPGYVIEVNPRASRTVPFLSKVTNI | 100 |
| IIENLPKVMDKYGISSL | 101 |
| EELRQEVKE | 102 |
| MTTNRLQVSLPGLDLKNPIIPASGCFGFGQEYAKYYDL | 103 |
| EAALEMYLAGASAIGVGTANFTNPYACP | 104 |
| AAQMDGAILVVAST | 105 |
| QERILEDINLQVTSGEVVS | 106 |
| IVSEIKLDWSEDEDKEVQKIAYKRQILAELGLD | 107 |
| LLKEFQELETFADF | 108 |
| AKLAEFELAGKP | 109 |
| LSGVPEAKDK | 110 |
| YKEWGMPAH | 111 |
| FLPFGVSPDFMDARINVLWASAPSTILPDTTYYAEEHPQREEL | 112 |
| TLWKESSANLLKAYDFSDEEIEDLLEKRLELDRRV | 113 |
| WSLLKATLILSVVNLSTSYLTEDIRVL | 114 |
| YQRVYRYLDKRVD | 115 |
| LKDSRTAKYHKRLQIVLF | 116 |
| ILDACPLVLDCRVDRIVEEDGICHIFAKILERLVAPE | 117 |
| LDEKGHFKNQLFAPTYFMGDG | 118 |
| MNIIEEIMTKLREDIRNIAII | 119 |
| VDELLKQSETLDARTELAERAMDSNDIEKERGITI | 120 |
| EVVDEVLELFIELGADDDQLDFPVVYASAINGTSSLSDDPADQE, | 121 |

and variants and analogs thereof.

Variants of the peptides of the present invention include substitution of one amino acid residue maximum per each nine amino acid residues in a peptide sequence, namely, peptides having about 90% or more identity are included within the scope of the present invention. According to some embodiments, sequences having at least about 95% identity to the peptides of the present invention are provided.

According to yet other embodiments the present invention provides a synthetic or recombinant peptide of 9-20 amino acids selected from the group of SEQ ID NOS: 26, 27, 28, 33, 34, 35, 38, 39, 42, 43, 46, 48, 53, 55, 56, 57, 66, 67, 70, 71, 74, 75, 76, 77, 78, 79, 80, 81, 82, 83, 87, 90, 91, 101, 102, 105, 106, 108, 109, 110, 111, 115 and 116.

According to yet other embodiments the present invention provides a synthetic or recombinant peptide of 21-50 amino acids selected from the group of SEQ ID NOS: 29, 30, 31, 32, 36, 37, 40, 41, 44, 45, 47, 49, 50, 51, 52, 54, 58, 59, 60, 61, 62, 63, 64, 65, 68, 69, 72, 73, 84, 85, 86, 88, 89, 92, 93, 94, 95, 96, 97, 98, 99, 100, 103, 104, 107, 112, 113, 114, 117, 118, 119, 120, 121.

According to yet other embodiments, at least one peptide multimer according to the invention is conjugated to at least one carbohydrate moiety. According to some embodiments the carbohydrate moiety is an S. pneumoniae polysaccharide or is derived from an S. pneumoniae polysaccharide.

According to some specific embodiments the present invention provides a conjugate comprising at least one peptide derived from an age-dependent S. pneumoniae protein and at least one moiety comprising one or more saccharide units. The peptidic and saccharide moieties may me connected directly or through a spacer or a linker. According to some embodiments the saccharide moiety is an S. pneumoniae polysaccharide or is derived from an S. pneumoniae capsular polysaccharide. According to some embodiments the S. pneumoniae polysaccharide is selected from the group consisting of: serotypes 1, 3, 4, 5, 6A, 6B, 7F, 9V, 14, 18C, 19A, 19F, and 23F.

According to some embodiments at least one peptide or peptide-multimer of the present invention is produced as part of a fusion protein comprising a carrier sequence, namely the peptides are inserted within a sequence of a carrier polypeptide or are fused to a free amino group or a free carboxy group of a carrier protein sequence, which, according to certain embodiments is a S. pneumoniae protein or fragment and according to other embodiments the carrier protein sequence serves as an adjuvant.

According to yet other embodiments, the at least one peptide or peptide multimer of the invention is conjugated to a carbohydrate moiety, in some specific embodiments to an S. pneumoniae polysaccharide moiety.

According to certain embodiments the carrier polypeptide is selected from the group consisting of: detoxified pneumolysin, hsp60 or a fragment thereof.

The present invention provides, according to another aspect, isolated polynucleotide sequences encoding a sequence comprising at least one peptide of SEQ ID numbers: 26-121.

According to some embodiments, a polynucleotide sequence encoding a peptide-multimer is provided.

According to some specific embodiments the polynucleotide sequence encodes a peptide-multimer selected from the group consisting of SEQ ID NO: 122, 124, 126, 128, and 130.

According to other specific embodiments the polynucleotide sequence encoding a peptide-multimer is selected from the group consisting of SEQ ID NO: 123, 125, 127, 129 and 131.

According to some embodiments the invention provides isolated polynucleotide sequences encoding a chimeric or fusion polypeptide comprising at least one peptide of SEQ ID numbers: 26-121.

Also provided by the present invention are vectors comprising polynucleotide sequences encoding peptide sequence as well as chimeric or fusion polypeptide comprising at least one peptide of SEQ ID numbers: 26-121, operably linked to one or more transcription control elements.

According to an additional aspect, the present invention provides a host cell comprising vectors comprising polynucleotide sequences encoding a chimeric or fusion polypeptide comprising at least one peptide of SEQ ID numbers: 26-121.

According to yet another aspect, the present invention provides vaccine compositions for immunization of a subject against S. pneumoniae comprising at least one synthetic or recombinant peptide of 9-50 amino acids derived from an age-dependent S. pneumoniae cell-wall or cell membrane protein.

According to other embodiments, a vaccine composition according to the present invention further comprises at least one additional antigenic moiety of S. pneumoniae, such as a peptide or protein sequence or a polysaccharide moiety.

According to some embodiments the vaccine composition further comprises an adjuvant. According to other embodiments the vaccine does not contain an adjuvant.

Pharmaceutically acceptable adjuvants include, but are not limited to: water in oil emulsion, lipid emulsion, and liposomes. According to specific embodiments the adjuvant is selected from the group consisting of: CCS/C®, Montanide®, alum, muramyl dipeptide, Gelvac®, chitin microparticles, chitosan, cholera toxin subunit B, labile toxin, AS21V, AS02V, Intralipid®, and Lipofundin®.

In some embodiments the vaccine is formulated for intramuscular, intranasal, oral, intraperitoneal, subcutaneous, topical, intradermal and transdermal delivery. In some embodiments the vaccine is formulated for intramuscular administration. In other embodiments the vaccine is formulated for oral administration. In yet other embodiments the vaccine is formulated for intranasal administration.

The present invention provides according to a further aspect a method for inducing an immune response and conferring protection against S. pneumoniae in a subject, comprising administering a vaccine composition comprising at least one synthetic or recombinant peptide of 9-50 amino acids derived from the sequence of a cell-wall or cell-membrane protein of S. pneumoniae associated with age-dependent immune response, and variants and analogs thereof. According to some preferred embodiments the composition comprises a peptide-multimer or a fusion polypeptide comprising at least one synthetic or recombinant peptide, variant or analog of 9-50 amino acids derived from the sequence of a cell-wall or cell-membrane protein of S. pneumoniae associated with age-dependent immune response.

The route of administration of the vaccine is selected from intramuscular, oral, intranasal, intraperitoneal, subcutaneous, topical, intradermal, and transdermal delivery. According to preferred embodiments the vaccine is administered by intramuscular, intranasal or oral routes.

According to a further aspect of the present invention, a composition comprising at least one synthetic or recombinant S. pneumoniae derived peptide of 9-50 amino acids, and variants, analogs, peptide-multimers and fusion polypeptides thereof, is used for protection against an S. pneumoniae infection in a subject.

Production of a peptide immunogen of 9-50 amino acids derived from an S. pneumoniae age-dependent protein, and of variants, analogs, peptide-multimers, conjugates and fusion polypeptides thereof, by isolated polynucleotide sequence according to the invention is also within the scope of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. represents gel-filtration analysis of the purified P21 polypeptide on an analytical Superdex 75 column pre-equilibrated with TN buffer, pH 8. The main peak with retention time of 11.19 min corresponds to monomer. To estimate the molecular mass, the column was calibrated with BSA (66 kDa), rat CNTF (22 kDa) and human leptin (16 kD).

FIG. 2. depicts an SDS-PAGE (12%) of lyophilized P21 dissolved in UPW applied at 2, 6, 20 μg per lane in the presence of (3-mercaptoethanol (ME).

FIG. 3. is a gel filtration presentation of purified lyophilized P22, dissolved in UPW, as analyzed on analytical Superdex 75 column equilibrated with TN buffer.

FIG. 4. SDS-PAGE (12%) of lyophilized P22 dissolved in UPW in presence of ME and applied at 2, 6, 20 μg per lane.

FIG. 5. SDS-PAGE (12%) in presence of ME testing the mini-expression of four induced (+) and non-induced (−) clones of P29 in the insoluble fraction of pLys cells suspended in 1 ml UPW and 20 microlitters were loaded on the gel.

FIG. 6. SDS-PAGE (12%) in presence of ME testing the mini-expression of four induced (+) and non-induced (−) clones of P28 in the insoluble fraction of pLys cells suspended in 1 ml UPW and 20 microlitters were loaded on the gel.

FIG. 7. SDS-PAGE (12%) in presence of ME testing the mini-expression of four induced (+) and non-induced (−) clones of P27 in the insoluble fraction of pLys cells suspended in 1 ml UPW and 20 microlitters were loaded on the gel.

### DETAILED DESCRIPTION OF THE INVENTION

Novel therapeutic strategies are necessary to counter the prevalence of antibiotic-resistant pneumococci and the limitations of currently available vaccines. Future discovery of therapeutic modalities requires a better understanding of the dynamic interplay between pathogen and host, which leads either to S. pneumoniae clearance or to disease development. It is suspected that inappropriate or altered immune responses underlie the switch from benign carriage to clinical disease. It has been observed in infants that the antibody response to S. pneumoniae increases with age and correlates negatively with morbidity. The development of a peptide-based universal vaccine against S. pneumoniae will prevent replacement carriage and diseases development, caused by serotypes not included in the vaccine, observed following immunization with the polysaccharide-based vaccine. Furthermore, such vaccine may be used in subjects previously immunized with the polysaccharide vaccine.

As previously described (WO 2003/082183), to identify proteins having vaccine potential a cell wall fraction was extracted from S. pneumoniae. About 150 proteins from the cell wall fraction were screened by 2D-Western blotting using sera obtained longitudinally from children attending day care centers and sera from healthy adult volunteers. About thirty proteins exhibited age-dependent antigenicity and are therefore denoted “age-dependent”. The sequences of the age-dependent proteins were determined and the proteins identified. SEQ ID NOs. 1-25 represent a non-limitative list of S. pneumoniae age-dependent proteins according to the invention:

phosphoglucomutase/phosphomannomutase family protein (Accession No. NP_{—}346006, SEQ ID NO:1); elongation factor G/tetracycline resistance protein (tetO), (Accession No. NP_{—}344811, SEQ ID NO:2); Aspartyl/glutamyl-tRNA amidotransferase subunit C (Accession No. NP_{—}344960, SEQ ID NO:3); L-lactate dehydrogenase (Accession No. NP_{—}345686, SEQ ID NO:4); glyceraldehyde 3-phosphate dehydrogenase (GAPDH), (Accession No. NP_{—}346439, SEQ ID NO:5); UDP-glucose 4-epimerase (Accession No. NP_{—}346261, SEQ ID NO:6); elongation factor Tu family protein (Accession No. NP 358192, SEQ ID NO:7); Bifunctional GMP synthase/glutamine amidotransferase protein (Accession No. NP_{—}345899, SEQ ID NO:8); glutamate dehydrogenase (Accession No. NP_{—}345769, SEQ ID NO:9); Elongation factor TS (Accession No. NP_{—}346622, SEQ ID NO:10); phosphoglycerate kinase (TIGR4) (Accession No. AAK74657, SEQ ID NO:11); 30S ribosomal protein S1 (Accession No. NP_{—}345350, SEQ ID NO:12); 6-phosphogluconate dehydrogenase (Accession No. NP_{—}357929, SEQ ID NO:13); aminopeptidase C (Accession No. NP_{—}344819, SEQ ID NO:14); carbamoyl-phosphate synthase (large subunit) (Accession No. NP_{—}345739, SEQ ID NO:15); PTS system, mannose-specific IIAB components (Accession No. NP_{—}344822, SEQ ID NO:16); 30S ribosomal protein S2 (Accession No. NP_{—}346623, SEQ ID NO:17); dihydroorotate dehydrogenase 1B (Accession No. NP_{—}358460, SEQ ID NO:18); aspartate carbamoyltransferase catalytic subunit (Accession No. NP_{—}345741, SEQ ID NO:19); elongation factor Tu (Accession No. NP_{—}345941, SEQ ID NO:20); Pneumococcal surface immunogenic protein A (PsipA) (Accession No. NP_{—}344634, SEQ ID NO:21); phosphoglycerate kinase (R6) (Accession No. NP_{—}358035, SEQ ID NO:22); ABC transporter substrate-binding protein (Accession No. NP_{—}344690, SEQ ID NO:23); endopeptidase 0 (Accession No. NP 346087, SEQ ID NO:24); Pneumococcal surface immunogenic protein C (PsipC) (Accession No. NP_{—}345081, SEQ ID NO:25), and variants and analogs thereof.

These proteins were observed to elicit cross-strain protection against lethal intranasal pneumococcal challenge in a mouse model (Ling et al., Clin Exp Immunol 2004, 138, 290-8). The proteins and antibodies raised against them were found to inhibit bacterial adhesion to cultured epithelial cells in vitro. Moreover, the antibodies produced against these proteins inhibited nasopharyngeal and lung colonization in vivo, suggesting that these proteins are involved in bacterial adhesion to the host.

In attempt to reduce protein load upon vaccination and to identify immunogenic peptides derived from the above age-dependent proteins, peptides, synthesized for example in a peptide array, are prepared and screened with sera obtained longitudinally from infants, healthy adults and from mice immunized with the intact age-dependent proteins.

It is now disclosed, that certain peptides derived from the above 25 age-dependent proteins of S. pneumoniae, lack sequence homology to human proteins, and possess high homology between all the currently sequenced strains of S. pneumoniae, retain the age-dependency characteristic in children, and can be used in improved vaccines against S. pneumoniae.

These peptides, alone, as part of multimeric constructs, conjugated to or mixed with a carrier protein, with additional antigenic moieties, and/or with an adjuvant, are effective in protecting subjects against infection with S. pneumoniae.

For convenience, certain terms employed in the specification, examples and claims are described herein.

The term “antigen presentation” means the expression of antigen on the surface of a cell in association with major histocompatibility complex class I or class II molecules (MHC-I or MHC-II) of animals or with the HLA-I and HLA-II of humans.

The term “immunogenicity” or “immunogenic” relates to the ability of a substance to stimulate or elicit an immune response. Immunogenicity is measured, for example, by determining the ability to produce antibodies specific for the substance. The presence of antibodies is detected by methods known in the art, for example using an ELISA assay, or immunoblotting. The term “antigenicity” or “antigenic” refer to a substance identified by an antibody or by the T cell receptor.

“Amino acid sequence”, as used herein, refers to an oligopeptide, peptide, polypeptide, or protein sequence, and fragment thereof, whether to naturally occurring or synthetic.

A “chimeric protein/polypeptide” or a “fusion protein/polypeptide” are used interchangeably and refer to an immunogenic peptide or peptides operatively linked to a polypeptide or protein.

A “peptide-multimer” refers to a construct comprising at least two covalently linked, immunogenic peptides according to the invention. The at least two peptides may be identical or different and may be derived from one or more S. pneumoniae proteins, and the peptide-multimer may include at least one sequence of a carrier protein or a protein fragment which is optional functionalized as an adjuvant.

### Synthetic Peptides

The peptides of the present invention may be synthesized chemically using methods known in the art for synthesis of peptides and polypeptides. These methods generally rely on the known principles of peptide synthesis; most conveniently, the procedures can be performed according to the known principles of solid phase peptide synthesis.

As used herein “peptide” indicates a sequence of amino acids linked by peptide bonds. A polypeptide is generally a peptide of about 51 and more amino acids.

Peptide analogs and peptidomimetics are also included within the scope of the invention as well as salts and esters of the polypeptides of the invention are encompassed.

A peptide analog according to the present invention may optionally comprise at least one non-natural amino acid and/or at least one blocking group at either the C terminus or N terminus. The design of appropriate “analogs” may be computer assisted.

The term “peptidomimetic” means that a peptide according to the invention is modified in such a way that it includes at least one non-peptidic bond such as, for example, urea bond, carbamate bond, sulfonamide bond, hydrazine bond, or any other covalent bond. The design of appropriate “peptidomimetic” may be computer assisted.

Salts and esters of the peptides of the invention are encompassed within the scope of the invention. Salts of the peptides and polypeptides of the invention are physiologically acceptable organic and inorganic salts. Functional derivatives of the peptides of the invention covers derivatives which may be prepared from the functional groups which occur as side chains on the residues or the N- or C-terminal groups, by means known in the art, and are included in the invention as long as they remain pharmaceutically acceptable, i.e., they do not destroy the antigenicity of the peptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “amino acid” refers to compounds, which have an amino group and a carboxylic acid group, preferably in a 1,2-1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanolanine), and synthetically derived α-amino acids, such as amino-isobutyric acid, norleucine, norvaline, homocysteine and homoserine. β-Alanine and γ-amino butyric acid are examples of 1,3 and 1,4-amino acids, respectively, and many others are well known to the art. Statine-like isosteres (a dipeptide comprising two amino acids wherein the CONH linkage is replaced by a CHOH), hydroxyethylene isosteres (a dipeptide comprising two amino acids wherein the CONH linkage is replaced by a CHOHCH₂), reduced amide isosteres (a dipeptide comprising two amino acids wherein the CONH linkage is replaced by a CH₂NH linkage) and thioamide isosteres (a dipeptide comprising two amino acids wherein the CONH linkage is replaced by a CSNH linkage) are also useful residues for this invention.

The amino acids used in this invention are those, which are available commercially or are available by routine synthetic methods. Certain residues may require special methods for incorporation into the peptide, and sequential, divergent or convergent synthetic approaches to the peptide sequence are useful in this invention. Natural coded amino acids and their derivatives are represented by three-letter codes according to IUPAC conventions. When there is no indication, the L isomer was used.

Conservative substitutions of amino acids as known to those skilled in the art are within the scope of the present invention, as long as antigenicity is preserved in the substituted peptide. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

Variants of the peptides of the present invention include substitution of one amino acid residue maximum per each nine amino acid residues in a peptide sequence, namely, peptides having about 90% or more identity are included within the scope of the present invention. According to some embodiments, sequences having at least about 95% identity to the peptides of the present invention are provided.

### Recombinant Production of Peptides and Polypeptides

The peptides and polypeptides of the present invention can be prepared by expression in an expression vector per se or as a chimeric protein. The methods to produce a chimeric or recombinant protein comprising one or more peptides derived from age-dependent proteins of S. pneumoniae, are known to those with skill in the art. A nucleic acid sequence encoding one or more polypeptide comprising at least one such peptide can be inserted into an expression vector for preparation of a polynucleotide construct for propagation and expression in host cells.

The term “expression vector” and “recombinant expression vector” as used herein refers to a DNA molecule, for example a plasmid or virus, containing a desired and appropriate nucleic acid sequences necessary for the expression of the recombinant polypeptides for expression in a particular host cell. As used herein “operably linked” refers to a functional linkage of at least two sequences. Operably linked includes linkage between a promoter and a second sequence located down stream of this promoter, for example an nucleic acid sequence of peptides described in present invention, wherein the promoter sequence facilitates and mediates transcription of the DNA sequence corresponding to the second sequence.

The regulatory regions necessary for transcription of the polypeptides can be provided by the expression vector. The precise nature of the regulatory regions needed for gene expression may vary among vectors and host cells. Generally, a promoter is required which is capable of binding RNA polymerase and promoting the transcription of an operably-associated nucleic acid sequence. Regulatory regions may include those 5′ non-coding sequences involved with initiation of transcription and translation, such as the Shine-Dalgarno sequence in E. Coli (the ribosomal binding site in the mRNA, generally located 8 basepairs upstream of the start codon) and initiation factors are also required to start translation the box Pribnow box TATAAT at −10 and the like. The non-coding region 3′ to the coding sequence may contain transcriptional termination regulatory sequences (TAA, TAG, or TGA), such as terminators and/or analogous once using Eukaryote expression systems. A translation initiation codon (ATG) may also be provided.

In order to clone the nucleic acid sequences into the cloning site of a vector, linkers or adapters providing the appropriate compatible restriction sites are added during synthesis of the nucleic acids. For example, a desired restriction enzyme site can be introduced into a fragment of DNA by amplification of the DNA by use of PCR with primers containing the desired restriction enzyme site. An alternative method is gene synthesis approaches which are most often based on a combination of organic chemistry and molecular biological techniques and entire genes may be synthesized “de novo”, without the need for precursor template DNA.

An expression construct comprising a peptide sequence operably associated with regulatory regions can be directly introduced into appropriate host cells for expression and production of peptide per se or as recombinant fusion protein. The expression vectors that may be used include but are not limited to plasmids, cosmids, phage, phagemids or modified viruses. Typically, such expression vectors comprise a functional origin of replication for propagation of the vector in an appropriate host cell, one or more restriction endonuclease sites for insertion of the desired gene sequence, and one or more selection markers.

The recombinant polynucleotide construct comprising the expression vector and a peptide according to the invention should then be transferred into a bacterial host cell where it can replicate and be expressed. This can be accomplished by methods known in the art. The expression vector is used with a compatible prokaryotic or eukaryotic host cell which may be, according to some embodiments, derived from bacteria, yeast, insects, mammals and humans.

Once expressed by the host cell, the peptide or peptide-multimer can be separated from undesired components by a number of protein purification methods. One such method uses a polyhistidine tag on the recombinant protein. A polyhistidine-tag consists in at least six histidine (His) residues added to a recombinant polypeptide, often at the N- or C-terminus. Polyhistidine-tags are often used for affinity purification of polyhistidine-tagged recombinant proteins that are expressed in E. coli or other prokaryotic expression systems. The bacterial cells are harvested by centrifugation and the resulting cell pellet can be lysed by physical means or with detergents or enzymes such as lysozyme. The raw lysate contains at this stage the recombinant polypeptide among several other proteins derived from the bacteria and are incubated with affinity media such as NTA-agarose, HisPur resin or Talon resin. These affinity media contain bound metal ions, either nickel or cobalt to which the polyhistidine-tag binds with micromolar affinity. The resin is then washed with phosphate buffer to remove proteins that do not specifically interact with the cobalt or nickel ion. The washing efficiency can be improved by the addition of 20 mM imidazole and the polypeptides are then usually eluted with 150-300 mM imidazole. The polyhistidine tag may be subsequently removed using restriction enzymes, endoproteases or exoproteases. Kits for the purification of histidine-tagged polypeptides can be purchased for example from Qiagen.

Another method is through the production of inclusion bodies, which are inactive aggregates of polypeptide that may form when a recombinant polypeptide is expressed in a prokaryote. While the cDNA may properly code for a translatable mRNA, the protein that results may not fold correctly, or the hydrophobicity of the sequence may cause the recombinant polypeptide to become insoluble. Inclusion bodies are easily purified by methods well known in the art. Various procedures for the purification of inclusion bodies are known in the art. In some embodiments the inclusion bodies are recovered from bacterial lysates by centrifugation and are washed with detergents and chelating agents to remove as much bacterial protein as possible from the aggregated recombinant protein. To obtain soluble polypeptide, the washed inclusion bodies are dissolved in denaturing agents and the released protein is then refolded by gradual removal of the denaturing reagents by dilution or dialysis. Purification of the protein is then performed, for example, using fractionation by charge or size on resin columns as known in the art.

Yet another method for the isolation of an expressed soluble untagged polypeptide involved its precipitation with increasing concentrations of ammonium sulfate followed by refolding and purification.

In E. coli an ATG, or occasionally a GTG, sequence must precede the gene coding sequence, for translation initiation. Thus the primary products of translation possess an N-terminal methionine residue. E. coli possesses enzymes which catalyse the efficient removal of the methionine residues from natural proteins when required, but these enzymes do not work with the same efficiency on recombinant polypeptides and therefore expressed proteins may possess an unnatural N-terminal methionine residue. The extra methionine residue at the N terminus of some of the recombinant proteins of the invention (for example SEQ ID NOs 1-25, 122, 124, 126, 128, 130), may be present in the final protein or may be removed at any stage of the expression, process or purification.

### Vaccine Formulation

The vaccines of the present invention comprise at least one immunogenic peptide derived from S. pneumoniae age-dependent proteins, and optionally, at least one adjuvant and/or an excipient. Formulation can contain a variety of additives, such as adjuvant, excipient, stabilizers, buffers, or preservatives. The vaccine can be formulated for administration in one of many different modes.

The choice of the adjuvant will be determined in part by the mode of administration of the vaccine. In a particular embodiment, the vaccine is formulated for parenteral administration, for example intramuscular administration. According to yet another embodiment the administration is orally.

According to yet another embodiment the administration is intradermal. Needles specifically designed to deposit the vaccine intradermally are known in the art as disclosed for example in U.S. Pat. No. 6,843,781 and U.S. Pat. No. 7,250,036 among others. According to other embodiments the administration is performed with a needleless injector.

According to one embodiment of the invention, the vaccine is administered intranasally. The vaccine formulation may be applied to the mucosal tissue of the nose in any convenient manner. However, it is preferred to apply it as a liquid stream or liquid droplets to the walls of the nasal passage. The intranasal composition can be formulated, for example, in liquid form as nose drops, spray, or suitable for inhalation, as powder, as cream, or as emulsion. Non-limiting examples of intranasal adjuvants include chitosan powder, PLA and PLG microspheres, QS-21, AS02V, calcium phosphate nanoparticles (CAP); mCTA/LTB (mutant cholera toxin E112K with pentameric B subunit of heat labile enterotoxin), and detoxified-E. Coli derived labile toxin.

In another embodiment of the invention, administration is oral and the vaccine may be presented, for example, in the form of a tablet or encased in a gelatin capsule or a microcapsule.

The formulation of these modalities is general knowledge to those with skill in the art.

The adjuvant used may also be, theoretically, any of the adjuvants known for peptide- or protein-based vaccines. For example: inorganic adjuvants in gel form (aluminium hydroxide/aluminium phosphate, calcium phosphate); bacterial adjuvants such as monophosphoryl lipid A and muramyl peptides; particulate adjuvants such as the so-called ISCOMS (“immunostimulatory complexes”), liposomes and biodegradable microspheres; adjuvants based on oil emulsions and emulsifiers such as IFA (“Incomplete Freund's adjuvant” (Stuart-Harris, 1969; Warren et al., 1986), SAF, saponines (such as QS-21), squalene/squalane; synthetic adjuvants such as non-ionic block copolymers, muramyl peptide analogs, synthetic lipid A, synthetic polynucleotides and polycationic adjuvants.

Liposomes provide another delivery system for antigen delivery and presentation. Liposomes are bilayered vesicles composed of phospholipids and other sterols surrounding a typically aqueous center where antigens or other products can be encapsulated. The liposome structure is highly versatile with many types range in nanometer to micrometer sizes, from about 25 nm to about 500 μm. Liposomes have been found to be effective in delivering therapeutic agents to dermal and mucosal surfaces. Liposomes can be further modified for targeted delivery by for example, incorporating specific antibodies into the surface membrane, or altered to encapsulate bacteria, viruses or parasites. The average survival time or half life of the intact liposome structure can be extended with the inclusion of certain polymers, for example polyethylene glycol, allowing for prolonged release in vivo. Liposomes may be unilamellar or multilamellar.

The vaccine composition may be formulated by: encapsulating an antigen or an antigen/adjuvant complex in liposomes to form liposome-encapsulated antigen and mixing the liposome-encapsulated antigen with a carrier comprising a continuous phase of a hydrophobic substance. If an antigen/adjuvant complex is not used in the first step, a suitable adjuvant may be added to the liposome-encapsulated antigen, to the mixture of liposome-encapsulated antigen and carrier, or to the carrier before the carrier is mixed with the liposome-encapsulated antigen. The order of the process may depend on the type of adjuvant used. Typically, when an adjuvant like alum is used, the adjuvant and the antigen are mixed first to form an antigen/adjuvant complex followed by encapsulation of the antigen/adjuvant complex with liposomes. The resulting liposome-encapsulated antigen is then mixed with the carrier. The term “liposome-encapsulated antigen” may refer to encapsulation of the antigen alone or to the encapsulation of the antigen/adjuvant complex depending on the context. This promotes intimate contact between the adjuvant and the antigen and may, at least in part, account for the immune response when alum is used as the adjuvant. When another is used, the antigen may be first encapsulated in liposomes and the resulting liposome-encapsulated antigen is then mixed into the adjuvant in a hydrophobic substance.

In formulating a vaccine composition that is substantially free of water, antigen or antigen/adjuvant complex is encapsulated with liposomes and mixed with a hydrophobic substance. In formulating a vaccine in an emulsion of water-in-a hydrophobic substance, the antigen or antigen/adjuvant complex is encapsulated with liposomes in an aqueous medium followed by the mixing of the aqueous medium with a hydrophobic substance. In the case of the emulsion, to maintain the hydrophobic substance in the continuous phase, the aqueous medium containing the liposomes may be added in aliquots with mixing to the hydrophobic substance.

In all methods of formulation, the liposome-encapsulated antigen may be freeze-dried before being mixed with the hydrophobic substance or with the aqueous medium as the case may be. In some instances, an antigen/adjuvant complex may be encapsulated by liposomes followed by freeze-drying. In other instances, the antigen may be encapsulated by liposomes followed by the addition of adjuvant then freeze-drying to form a freeze-dried liposome-encapsulated antigen with external adjuvant. In yet another instance, the antigen may be encapsulated by liposomes followed by freeze-drying before the addition of adjuvant. Freeze-drying may promote better interaction between the adjuvant and the antigen resulting in a more efficacious vaccine.

Formulation of the liposome-encapsulated antigen into a hydrophobic substance may also involve the use of an emulsifier to promote more even distribution of the liposomes in the hydrophobic substance. Typical emulsifiers are well-known in the art and include mannide oleate (Arlacel™ A), lecithin, Tween™ 80, Spans™ 20, 80, 83 and 85. The emulsifier is used in an amount effective to promote even distribution of the liposomes.

According to some embodiments of the preset invention, the adjuvant CCS/C® is included in the vaccine formulation. CCS/C® is a synthetic polycationic sphingolipid derived from D-erythro ceramide to which spermine is covalently attached, thereby forming Ceramide Carbamoyl Spermine (CCS). CCS mixed with cholesterol (CCS/C) self-assembles into liposomes known as VaxiSome. Based on its structure and components (ceramide, CO2 and spermine), CCS is predicted to be biocompatible and biodegradable. In vitro and in vivo studies suggest that the CCS/C formulation upregulates levels of CD40 and B7 co-stimulatory molecules, which are essential in antigen presentation and T-helper cell activation. As a result, VaxiSome is a potent liposomal adjuvant for stimulating enhanced immune responses via the Th1 and Th2 pathways.

Microparticles and nanoparticles employ small biodegradable spheres which act as depots for vaccine delivery. The major advantage that polymer microspheres possess over other depot-effecting adjuvants is that they are known as safe and have been approved by the Food and Drug Administration in the US for use in human medicine as suitable sutures and for use as a biodegradable drug delivery system (Langer R. Science. 1990; 249(4976):1527-33). The rates of copolymer hydrolysis are very well characterized, which in turn allows for the manufacture of microparticles with sustained antigen release over prolonged periods of time (O'Hagen, et al., Vaccine. 1993; 11(9):965-9).

Parenteral administration of microparticles elicits long-lasting immunity, especially if they incorporate prolonged release characteristics. The rate of release can be modulated by the mixture of polymers and their relative molecular weights, which will hydrolyze over varying periods of time. Without wishing to be bound to any theory, the formulation of different sized particles (1 μm to 200 μm) may also contribute to long-lasting immunological responses since large particles must be broken down into smaller particles before being available for macrophage uptake. In this manner a single-injection vaccine could be developed by integrating various particle sizes, thereby prolonging antigen presentation and greatly benefiting livestock producers.

Another adjuvant for use with an immunogen of the present invention is an emulsion. A contemplated emulsion can be an oil-in-water emulsion or a water-in-oil emulsion. In addition to the peptide or peptide-multimer, such emulsions comprise an oil phase of squalene, squalane, peanut oil or the like as are well known, and a dispersing agent. Non-ionic dispersing agents are preferred and such materials include mono- and di-C₁₂-C₂₄-fatty acid esters of sorbitan and mannide such as sorbitan mono-stearate, sorbitan mono-oleate and mannide mono-oleate.

Such emulsions are for example water-in-oil emulsions that comprise squalene, glycerol and a surfactant such as mannide mono-oleate (Arlacel™ A), optionally with squalane. Alternative components of the oil-phase include alpha-tocopherol, mixed-chain di- and tri-glycerides, and sorbitan esters. Well-known examples of such emulsions include Montanide™ ISA-720, and Montanide™ ISA 703 (Seppic, Castres, France. Other oil-in-water emulsion adjuvants include, for example, those disclosed in WO 95/17210 and EP 0 399 843.

The use of small molecule adjuvants is also contemplated herein. One type of small molecule adjuvant useful herein is a 7-substituted-8-oxo- or 8-sulfo-guanosine derivative described in U.S. Pat. No. 4,539,205, U.S. Pat. No. 4,643,992, U.S. Pat. No. 5,011,828 and U.S. Pat. No. 5,093,318. 7-allyl-8-oxoguanosine(loxoribine) has been shown to be particularly effective in inducing an antigen specific response.

A useful adjuvant includes monophosphoryl lipid A (MPL®), 3-deacyl monophosphoryl lipid A (3D-MPL®). The adjuvant contains three components extracted from bacteria: monophosphoryl lipid (MPL) A, trehalose dimycolate (TDM) and cell wall skeleton (CWS) (MPL+TDM+CWS) in a 2% squalene/Tween™ 80 emulsion. This adjuvant can be prepared by the methods taught in GB 2122204B. Other compounds are structurally related to MPL® adjuvant called aminoalkyl glucosamide phosphates (AGPs) such as RC-529™ {2-[(R)-3-tetra-decanoyloxytetradecanoylamino]-ethyl-2-deoxy-4-O-phosphon-o-3-O—[(R)-3-tetradecanoyloxytetra-decanoyl]-2-[(R)-3-tetra-decanoyloxytet-radecanoyl-amino]-p-D-glucopyranoside triethylammonium salt, described for example is U.S. Pat. No. 6,355,257 and U.S. Pat. No. 6,303,347; U.S. Pat. No. 6,113,918; and U.S. Publication No. 03-0092643).

Further contemplated adjuvants include synthetic oligonucleotide adjuvants containing the CpG nucleotide motif one or more times (plus flanking sequences). The adjuvant designated QS21, available from Aquila Biopharmaceuticals, Inc., is an immunologically active saponin fractions having adjuvant activity derived from the bark of the South American tree Quillaja Saponaria Molina (e.g. Quil™ A), and the method of its production is disclosed in U.S. Pat. No. 5,057,540. Derivatives of Quil™ A, for example QS21 (an HPLC purified fraction derivative of Quil™ A also known as QA21), and other fractions such as QA17 are also disclosed. Semi-synthetic and synthetic derivatives of Quillaja Saponaria Molina saponins are also useful, such as those described in U.S. Pat. No. 5,977,081 and U.S. Pat. No. 6,080,725. The adjuvant denominated MF59 available from Chiron Corp. is described in U.S. Pat. No. 5,709,879 and U.S. Pat. No. 6,086,901.

Muramyl dipeptide adjuvants are also contemplated and include N-acetyl-muramyl-L-threonyl-D-isoglutamine (thur-MDP), N-acetyl-nor-muramyl-L-alanyl-D-isoglutamine [CGP 11637, referred to as nor-MDP], and N-acetylmuramyl-L-alanyl-D-isoglutaminyl-L-alanine-2-(1′-2′-dipalmityol-s-n-glycero-3-hydroxyphosphoryloxy) ethylamine [(CGP) 1983A, referred to as MTP-PE]. The so-called muramyl dipeptide analogues are described in U.S. Pat. No. 4,767,842.

Other adjuvant mixtures include combinations of 3D-MPL and QS21 (EP 0 671 948 B1), oil-in-water emulsions comprising 3D-MPL and QS21 (WO 95/17210, PCT/EP98/05714), 3D-MPL formulated with other carriers (EP 0 689 454 B1), QS21 formulated in cholesterol-containing liposomes (WO 96/33739), or immunostimulatory oligonucleotides (WO 96/02555). Adjuvant SBAS2 (now ASO₂) available from SKB (now Glaxo-SmithKline) contains QS21 and MPL in an oil-in-water emulsion is also useful. Alternative adjuvants include those described in WO 99/52549 and non-particulate suspensions of polyoxyethylene ether (UK Patent Application No. 9807805.8).

The use of an adjuvant that contains one or more agonists for toll-like receptor-4 (TLR-4) such as an MPL® adjuvant or a structurally related compound such as an RC-529® adjuvant or a Lipid A mimetic, alone or along with an agonist for TLR-9 such as a non-methylated oligo deoxynucleotide-containing the CpG motif is also optional.

A heat-shock protein (hsp), fragment or peptide is also an optional adjuvant, as a carrier protein or peptide, in a mixture, or as part of a fusion polypeptide expressed or synthesized together with at least one peptide according to the invention. For example, U.S. Pat. Nos. 5,736,146 and 5,869,058 provide peptides derived from human and E. coli heat-shock protein 60 (hsp60) as carriers for vaccination against viral and bacterial pathogens. Defined peptides present uniquely effective characteristics in conjugate vaccines due to the following reasons:
i. Hsp60 epitopes provide natural T-cell help; Humans are born with a high frequency of T cells responsive to hsp60, so no induction is needed and youngsters respond.
ii. Defined hsp60-peptide conjugates function as built-in adjuvants activating innate TLR-4 receptors on antigen presenting cells (APCs); the hsp60-conjugate vaccine administered in aqueous solution serves as its own adjuvant.
iii. Defined hsp60-peptide conjugates do not induce the production of competing antibodies and therefore do not suppress vaccination responses, even with multiple administrations.
iv. Boosting to the hsp60-epitope occurs naturally, since hsp60 is up-regulated at the site of any immune response (infection or tumor); the vaccination effect does not decline for prolonged periods. Immune memory is robust and effective.

Detoxified pneumolysin, known as a carrier protein and as an adjuvant (for example Michon et al., Vaccine, 18, 1732-1741, 1998), or fragment or analog thereof, can be also used in conjunction or conjugation of the peptides of the present invention.

Another type of adjuvant mixture comprises a stable water-in-oil emulsion further containing aminoalkyl glucosamine phosphates such as described in U.S. Pat. No. 6,113,918. An exemplary aminoalkyl glucosamine phosphates is the molecule known as RC-529 {(2-[(R)-3-tetradecanoyloxytetradecanoylamino]ethyl 2-deoxy-4-O-phosphono-3-O—[(R)-3-tetradecanoyl oxy-tetradecanoyl]-2-[(R)-3-tetradecanoyloxytetra-decanoylamino]-p-D-glucopyranoside triethylammonium salt)}. A preferred water-in-oil emulsion is described in WO 99/56776.

Adjuvants are utilized in an adjuvant amount, which can vary with the adjuvant, host animal and immunogen. Typical amounts can vary from about 1 μg to about 1 mg per immunization. Those skilled in the art know that appropriate concentrations or amounts can be readily determined.

Vaccine compositions comprising an adjuvant based on oil in water emulsion is also included within the scope of the present invention. The water in oil emulsion may comprise a metabolisable oil and a saponin, such as for example as described in U.S. Pat. No. 7,323,182.

According to several embodiments, the vaccine compositions of the present invention may contain one or more adjuvants, characterized in that it is present as a solution or emulsion which is substantially free from inorganic salt ions, wherein said solution or emulsion contains one or more water soluble or water-emulsifiable substances which is capable of making the vaccine isotonic or hypotonic. The water soluble or water-emulsifiable substances may be, for example, selected from the group consisting of: maltose; fructose; galactose; saccharose; sugar alcohol; lipid; and combinations thereof.

The peptides, peptide-multimers, polypeptides and fusion proteins of the present invention comprise according to several specific embodiments a proteosome adjuvant. The proteosome adjuvant comprises a purified preparation of outer membrane proteins of meningococci and similar preparations from other bacteria. These proteins are highly hydrophobic, reflecting their role as transmembrane proteins and porins. Due to their hydrophobic protein-protein interactions, when appropriately isolated, the proteins form multi-molecular structures consisting of about 60-100 nm diameter whole or fragmented membrane vesicles. This liposome-like physical state allows the proteosome adjuvant to act as a protein carrier and also to act as an adjuvant.

Vaccine compositions comprising different immunogenic peptides can be produced by mixing or linking a number of different peptides according to the invention with or without an adjuvant. In addition, an immunogenic peptide according to the present invention may be included in a vaccine composition comprising any other S. pneumoniae protein or protein fragment, including mutated proteins such as detoxified pneumolysin, or they can be linked to or produced in conjunction with any such S. pneumoniae protein or protein fragment.

Vaccine compositions according to the present invention may include, for example, influenza polypeptides or peptide epitopes, conjugated with or coupled to at least one immunogenic S. pneumoniae peptide according to the invention.

The antigen content is best defined by the biological effect it provokes. Naturally, sufficient antigen should be present to provoke the production of measurable amounts of protective antibody. A convenient test for the biological activity of an antigen involves the ability of the antigenic material undergoing testing to deplete a known positive antiserum of its protective antibody. The result is reported in the negative log of the LD₅₀ (lethal dose, 50%) for mice treated with virulent organisms which are pretreated with a known antiserum which itself was pretreated with various dilutions of the antigenic material being evaluated. A high value is therefore reflective of a high content of antigenic material which has tied up the antibodies in the known antiserum thus reducing or eliminating the effect of the antiserum on the virulent organism making a small dose lethal. It is preferred that the antigenic material present in the final formulation is at a level sufficient to increase the negative log of LD₅₀ by at least 1 preferably 1.4 compared to the result from the virulent organism treated with untreated antiserum. The absolute values obtained for the antiserum control and suitable vaccine material are, of course, dependent on the virulent organism and antiserum standards selected.

The following method may be also used to achieve the ideal vaccine formulation: starting from a defined antigen, which is intended to provoke the desired immune response, in a first step an adjuvant matched to the antigen is found, as described in the specialist literature, particularly in WO 97/30721. In a next step the vaccine is optimized by adding various isotonic-making substances as defined in the present inventions, preferably sugars and/or sugar alcohols, in an isotonic or slightly hypotonic concentration, to the mixture of antigen and adjuvant, with the composition otherwise being identical, and adjusting the solution to a physiological pH in the range from pH 4.0 to 10.0, particularly 7.4. Then, in a first step the substances or the concentration thereof which will improve the solubility of the antigen/adjuvant composition compared with a conventional, saline-buffered solution are determined. The improvement in the solubility characteristics by a candidate substance is a first indication that this substance is capable of bringing about an increase in the immunogenic activity of the vaccine.

Since one of the possible prerequisites for an increase in the cellular immune response is increased binding of the antigen to APCs, in a next step an investigation can be made to see whether the substance leads to an increase of this kind. The procedure used may be analogous to that described in the definition of the adjuvant, e.g. incubating APCs with fluorescence-labelled peptide or protein, adjuvant and isotonic-making substance. An increased uptake or binding of the peptide to APCs brought about by the substance can be determined by comparison with cells which have been mixed with peptide and adjuvant alone or with a peptide/adjuvant composition which is present in conventional saline buffer solution, using flow cytometry.

Vaccine compositions according to the present invention may include at least one carbohydrate moiety, for example at least one S. Pneumoniae capsular polysaccharide. The at least one carbohydrate moiety may be conjugated to a peptide or multimer according to the invention or may be mixed with the peptide/multimer composition. A non limitative list of carbohydrate moieties/polysaccharides include: 1, 3, 4, 5, 6A, 6B, 7F, 9V, 14, 18C, 19A, 19F, and 23F.

Conjugates according to the present invention may include at least one antigenic peptide derived from S. pneumoniae age-dependent protein covalently coupled to another moiety (e.g. protein, peptide, carbohydrate).

The peptide/peptide-multimer and the second moiety may be linked directly via a covalent bond through a bifunctional linker and/or through a spacer. The spacer may be used to allow distance between the peptide-multimer moiety and the other moiety (for example polysaccharide).

Conjugates according to the present invention may be formed, directly or through a linker, between any functional group of the peptide/polypeptide and a functional group of the other moiety to be conjugated. The optional connective linker may be of varied lengths and conformations comprising any suitable chemistry including but not limited to amine, amide, carbamate, thioether, oxyether, sulfonamide bond and the like. Non-limiting examples for such linkers include amino acids, sulfone amide derivatives, amino thiol derivatives and amino alcohol derivatives.

According to certain embodiments the linker comprises a cleavable sequence. According to one embodiment the cleavable linker is cleaved by intracellular enzymes. According to a specific embodiment the cleavable linker comprises a protease specific cleavable sequence.

For conjugation with carbohydrates, both direct and indirect conjugation requires chemical activation of the carbohydrate moiety prior to derivatisation (for example U.S. Pat. No. 5,651,971).

There are many conjugation reactions known in the prior art that have been employed for covalently linking polysaccharides to polypeptides in order to produce a polysaccharide-polypeptide conjugate. Three of the more commonly employed methods include: 1) reductive amination, wherein the aldehyde or ketone group on one component of the reaction reacts with the amino or hydrazide group on the other component, and the C—N double bond formed is subsequently reduced to C—N single bond by a reducing agent; 2) cyanylation conjugation, wherein the polysaccharide is activated either by cyanogens bromide (CNBr) or by 1-cyano-4-dimethylammoniumpyridinium tetrafluoroborate (CDAP) to introduce a cyanate group to the hydroxyl group, which forms a covalent bond to the amino or hydrazide group upon addition of the protein component; and 3) a carbodiimide reaction, wherein carbodiimide activates the carboxyl group on one component of the conjugation reaction, and the activated carbonyl group reacts with the amino or hydrazide group on the other component. These reactions are also frequently employed to activate the components of the conjugate prior to the conjugation reaction.

A carbohydrate or polysaccharide moiety may be conjugated to the polypeptide directly or via a linker. Linkage via a linker group may be made using any known procedure, for example, the procedures described in U.S. Pat. No. 4,882,317 and U.S. Pat. No. 4,695,624. Suitable linkers include carbonyl, adipic acid, B-propionamido (WO 00/10599), nitrophenyl-ethylamine, haloacyl halides (U.S. Pat. No. 4,057,685), glycosidic linkages (U.S. Pat. No. 4,673,574; U.S. Pat. No. 4,761,283; U.S. Pat. No. 4,808,700), 6-aminocaproic acid (U.S. Pat. No. 4,459,286), ADH (U.S. Pat. No. 4,965,338), C4 to Ci2 moieties (U.S. Pat. No. 4,663,160), etc. After conjugation of the polysaccharide to the polypeptide, the polysaccharide-polypeptide conjugate may be purified by a variety of techniques known in the art. One goal of the purification step is to remove the unbound polysaccharide and/or polypeptide from the polysaccharide-polypeptide conjugate. Methods for purification include e.g. ultrafiltration in the presence of ammonium sulfate, size exclusion chromatography, density gradient centrifugation, and hydrophobic interaction chromatography.

In an embodiment of the present invention, the composition may comprise two or more peptide/polypeptide-polysaccharide conjugates. In another embodiment, the composition comprises two or more peptide/polypeptide-polysaccharide conjugates, wherein the polysaccharide moieties are derived from different serotypes of the same bacteria, especially of different S. pneumoniae serotypes. Methods for combining several polysaccharide-polypeptide conjugates to multivalent compositions are well known in the art and are described e.g. in WO 2003/51392.

The efficiency of the formulation may optionally also be demonstrated by the cellular immune response by detecting a “delayed-type hypersensitivity” (DTH) reaction in immunized animals. Finally, the immunomodulatory activity of the formulation is measured in animal tests.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### Identification of Peptides Derived from Age-Dependent Proteins of S. pneumoniae

Analysis of sequence homology was performed using the BLAST software (http://blast.ncbi.nlm.nih.gov/Blast.cgi). Each of the proteins of SEQ ID NOS 1-25 was compared to the human genome using the program: “human build protein (previous build 35.1)” database and “BLASTP: compare protein sequence”. Area with less then nine amino acid homology sequences was define as non homology sequences. To insure that the sequences are streptococcus pneumoniae origin each non homology sequence was then compared to the protein in all Streptococcus pneumoniae strains sequenced as of October 2008-March 2009 (http://www.ncbi.nlm.nih.gov/sutils/genom_table.cgi). Non identical amino acids among the available S. pneumoniae sequences were removed from the non-human sequence homology peptides dividing those sequences to more peptides having decreases homology to human sequences and high homology to many S. pneumoniae strains.

The identified peptide sequences SEQ ID NOs. 26-121 of Table 1, have little or no homology to human sequences and retain 100% homology to all Streptococcus pneumoniae strains (NCBI, March 2009).

TABLE 1

| | | SEQ |
|---|---|---|
| Protein of origin & | | ID |
| Accession No. | Peptide | NO: |
| phosphoglucomutase- | AVAYLVETEGASAGVMI | 26 |
| phosphomannomutase family | MKEKAMEVPAIK | 27 |
| protein | YYVDTITDVVRAEIGID | 28 |
| NP 346006 | ASHNPALDNGIKFFGGDGFKLDDEKEAEIEALLDAEEDTLP | 29 |
| (SEQ ID NO: 1) | IGLAFDGDSDRLIAVDENGDIVDGDKIMYIIGKYLS | 30 |
| | TGKSLSELAAEVTIYPQKLVNIRVEN | 31 |
| | IIEKMEEEMAGNGRILVRPSGTEPLLRVMAEAPT | 32 |
| Elongation factor G | TAQWNNHRV | 33 |
| NP_344811 | LDIPAIKGINPDTD | 34 |
| (SEQ ID NO: 2) | GSYHDVDSSETAFKIAASL | 35 |
| | HKIGETHEGASQMDWMEQEQE | 36 |
| | LMMKYLEGEEITNEELKAGIRKATINVEFFPVLC | 37 |
| aspartyl glutamyl-tRNA | RDRLFKNVPE | 38 |
| amidotransferase subunit | DNYYIKVPAILD | 39 |
| C | ETAAFATTLSKIVDMVELLGEVDTTGVAPTTTMADRKTVL | 40 |
| NP_344960 | RPDVAEEG | |
| (SEQ ID NO: 3) | MKITQEEVTHVANLSKLRFSE | 41 |
| L-lactate dehydrogenase | HEKAVGDALDLSHAL | 42 |
| NP 345686 | SIVTQVVESGFKGIFLVA | 43 |
| (SEQ ID NO: 4) | STKQHKKVILVGDGAVGSSYAFALVNQGIAQELGIIEIPQ | 44 |
| | SPKKIYAAQYSDCADADLVVITAGAPQKPGETRLDLVGKN | 45 |
| | LAIN | |
| glyceraldehyde-3- | VLDGTETVISG | 46 |
| phosphate dehydrogenase | YTGDQMILDGPHRGGDLRRARAGAANIVPN | 47 |
| NP 346439 | | |
| (SEQ ID NO: 5) | | |
| UDP-glucose 4- | ALRKVFEQESIDAA | 48 |
| epimerase | VELLNLGKEVIIVDNLSNSS | 49 |
| NP 346261 | LVLDRIEAITGIRPVFYELDVCDK | 50 |
| (SEQ ID NO: 6) | VMYYKNNIMSTLALVEVMSEFNVKKI | 51 |
| | NPYGYTKVMLEQILKDVHVADSEWSIALLR | 52 |
| Bifunctional GMP | DGSFDIDPEIFELGI | 53 |
| synthase/glutamine | MQFELIEPLNTLYKDEVRALGTELGMPDHIVWRQ | 54 |
| amidotransferase protein | | |
| NP 345899 | | |
| (SEQ ID NO: 8) | | |
| glutamate dehydrogenase | MTSAKEYIQSVFETVK | 55 |
| NP 345769 | QGVICVSEGANMPS | 56 |
| (SEQ ID NO: 9) | LDAIKVYKENGI | 57 |
| | RNGHEAEFLQAVEEFFNTLEPVFEKHPEYIEENILARITEPE | 58 |
| | R | 59 |
| | MRFCQSFMTELQKHIGPSLDVPAGDIGVGGREIGYLYGQY | 60 |
| | KRLNQFD | |
| | GVLTGKPLGFGGSLIRPEATGYGL | 60 |
| | YYTEEMLKANGNSFAGKKVVISGSGNVAQYALQKATELG | 62 |
| | ATVIS | 63 |
| | SDSNGYVIDENGIDFDLLVDVKEKRRARLTEYAAEKATAT | 64 |
| | YH | 65 |
| | YAGNYDIALPCATQNEINGEAAKRLV | |
| | YGPAKAANAGGVAVSALEMSQNSLRLSWTREEVD | |
| | RLKDIMTNIFNTAKTTSETYGLDKDYLAGANIAAFENVAN | |
| | AMIAQG | |
| Elongation factor Ts NP | NDFEAEVAATMAAALNN | 66 |
| 346622 | | |
| (SEQ ID NO: 10) | | |
| Phosphoglycerate kinase | LEDGQVLLVENTRYEDVDG | 67 |
| NP 358035 | DKAGKSLAPVAADLAAKLGQDVVFPGVTRGAELEAAIN | 68 |
| (SEQ ID NO: 22) | KESKNDPELGKYWASLGDGIF | 69 |
| 30S ribosomal protein S2 | DAVAVEAEFAA | 70 |
| NP 346623 | ETQADSIEEIVEVVEGDNA | 71 |
| (SEQ ID NO: 17) | RLEKFLGGIEDMPRIPDVMYVVDPHKEQIAVKEAKKLGIPV | 72 |
| | VAMVDTN | |
| | DPDDIDVIIPANDDAIRAVKLITAKLADAIIEGRQG | 73 |
| 6phosphogluconate | IRRNEELANSG | 74 |
| dehydrogenase | LSRKDDEGQDGPIVDYIL | 75 |
| NP 357929 | LDVTAKYQQAVRDIVALA | 76 |
| (SEQ ID NO: 13) | PGTDATIQALLPHL | 77 |
| | QDGPIVDYIL | 78 |
| | VHASKVLPKPAAF | 79 |
| | GDKAELIEK | 80 |
| | AQLRVASKENNWNLPFAD | 81 |
| | VPVPTFSAAITYFDSYRSAD | 82 |
| | RKDKEGTFHYSW | 83 |
| | ESRGYTVAIYNRSKEKTEDVIACHPEKNFVPSYDVES | 84 |
| | DVLEEISAKAPEDGKPCVTYIGPDGA | 85 |
| | AESYDLMQHLLGLSAEDMAEIFT | 86 |
| Aminopeptidase C | MNAIQESFT | 87 |
| NP 344819 | KLFANYEANVKYQAIENAASHNGIFAALE | 88 |
| (SEQ ID NO: 14) | KWKVENSWGDKVGTDGYFVASDAWMDEYTYQIVVRKEL | 89 |
| | LTAEEQAAYGAE | |
| Carbamoyl phosphate | SFKYYVIEVNP | 90 |
| synthase large subunit | GVHHNEIPELA | 91 |
| NP 345739 | RTDIQKIMVIGSGPIIIGQAAEFDYAGTQAC | 92 |
| (SEQ ID NO: 15) | SLKEEGYEVVLVNSNPATIMTDKEIADKVYIEPITLEFVTRI | 93 |
| | LRKE | 94 |
| | PDALLPTLGGQTGLNMAMELSKNGILDEL | 95 |
| | SAIDQAEDRDLFKQLMEELEQPIPESEIVNTVEEA | 96 |
| | QTMSDYENQMLRDASLSIIRALKIEGGCNVQLALDP | 97 |
| | TAKLNGFSDRKIAELWGTTDD | 98 |
| | VRQLRLENKIVPVYKMVDTCAAEFDSETPYFYSTYGWENE | 99 |
| | SI | |
| | SDKESVLVLGSGPIRIGQGVEFDYATVHSVKAIQAAGYEAI | 100 |
| | VLIPGYVIEVNPRASRTVPFLSKVTNI | |
| dihydroorotate | IIENLPKVMDKYGISSL | 101 |
| dehydrogenase 1B | EELRQEVKE | 102 |
| NP 358460 | MTTNRLQVSLPGLDLKNPIIPASGCFGFGQEYAKYYDL | 103 |
| (SEQ ID NO: 18) | EAALEMYLAGASAIGVGTANFTNPYACP | 104 |
| Elongation factor Tu NP | AAQMDGAILVVAST | 105 |
| 345941 | | |
| (SEQ ID NO: 20) | | |
| ABC transporter, ATP- | QERILEDINLQVTSGEVVS | 106 |
| binding protein NP- | IVSEIKLDWSEDEDKEVQKIAYKRQILAELGLD | 107 |
| 344690 | | |
| (SEQ ID NO: 23) | | |
| endopeptidase O | LLKEFQELETFADF | 108 |
| NP 346087 | AKLAEFELAGKP | 109 |
| (SEQ ID NO: 24) | LSGVPEAKDK | 110 |
| | YKEWGMPAH | 111 |
| | FLPFGVSPDFMDARINVLWASAPSTILPDTTYYAEEHPQRE | 112 |
| | EL | |
| | TLWKESSANLLKAYDFSDEEIEDLLEKRLELDRRV | 113 |
| | WSLLKATLILSVVNLSTSYLTEDIRVL | 114 |
| Hypothetical protein | YQRVYRYLDKRVD | 115 |
| SP_0565 | | |
| NP 345081 | | |
| (SEQ ID NO: 25) | | |
| Hypothetical protein | LKDSRTAKYHKRLQIVLF | 116 |
| SP_0087 | ILDACPLVLDCRVDRIVEEDGICHIFAKILERLVAPE | 117 |
| NP 344634 | LDEKGHFKNQLFAPTYFMGDG | 118 |
| (SEQ ID NO: 21) | | |
| elongation factor Tu | MNIIEEIMTKLREDIRNIAII | 119 |
| family protein | VDELLKQSETLDARTELAERAMDSNDIEKERGITI | 120 |
| NP 358192 | EVVDEVLELFIELGADDDQLDFPVVYASAINGTSSLSDDPA | 121 |
| (SEQ ID NO: 7) | DQE | |

### Example 2

### Screening the Peptides

Peptide arrays and peptide libraries (reviewed for example in Reimer et al., Curr. Opin. Biotech. 2002, 13, 315-320), are used to synthesize peptides of table 1 and derivatives and analogs of these peptides. The peptides are synthesized using different linkers, matrixes and absorption methods, using methods known in the art (for example US 2002/0006672; Gaseitsiwe et al., Plos One 3, e3840, 1-8, 2008; Büssow et al., Am J Pharmacogenomics 2001; 1, 1-7; Andresen et al., Proteomics 6, 1376-1384, 2006). Peptides are obtained for screening either in a solution or absorbed or linked to a matrix. The peptide arrays are screened using sera obtained from infants at various ages as described for example in Ling et al., Clin Exp Immunol 2004. 138, 290-8.

A peptide list has been designed from the bacterial cell wall proteins with age-dependent antigenicity from protein domains with low homology to human proteins, and used to synthesize microarrays using methods known in the art. Typical peptide-array includes positive as well as negative peptide controls. For example, as a negative control the peptide MAAGAAEAAVAAVEE (SEQ ID NO:132) derived from Homo sapiens glutaredoxin 3, Accession number NP_{—}006532, and as a positive control the immunogenic peptide DNVLDHLTGRSCQ (SEQ ID NO:133) derived from pertussis toxin. An exemplary peptide-array includes 15 amino acid overlapping peptides (step of 2 amino acids between each overlapping peptide).

According to one specific method sera are collected longitudinally from healthy children attending day-care centers at different ages (for example 18, 30 and 42 months). Starting at 12 months of age, nasopharyngeal swabs are taken from the children on a bimonthly schedule over the 2.5 years of the study. Pneumococcal isolates are characterized by inhibition with optochin and a positive slide agglutination test (Phadebact, Pharmacia Diagnostics). In addition, sera are collected from healthy adults.

An increase in the antibody response to a peptide derived from a bacterial protein which coincides with the diminution in morbidity described in children indicates that the peptide retained the “age-dependent” characteristic of the bacterial protein and it is further synthesized and tested for its ability to elicit protection against S. pneumoniae. The identified peptides are also used as basis to design and synthesize chimeric genes and chimeric polypeptides containing multiple “age-dependent” peptides. The chimeric polypeptides are purified and tested for their immunogenicity and vaccine potential in animal model(s).

### Example 3

### Construction of Fusion Polypeptides and Peptide Multimers

Artificial genes encoding polypeptides comprising peptide sequences selected to be immunogenic and age dependent with or without carrier polypeptides, are constructed to encode chimeric proteins of up to 900 amino acids. The structure of the chimeric proteins is constructed to minimize homology to human sequences based on potential neoantigens at the fusion junction of peptides in the construct.

One set of constructs comprises 2-10 different peptides of 9-12 amino acids long, each in 1-4 repeats, with a spacer of 0-3 Glycine or Alanine residues between each peptide, and a detoxified pneumolysin as a carrier protein.

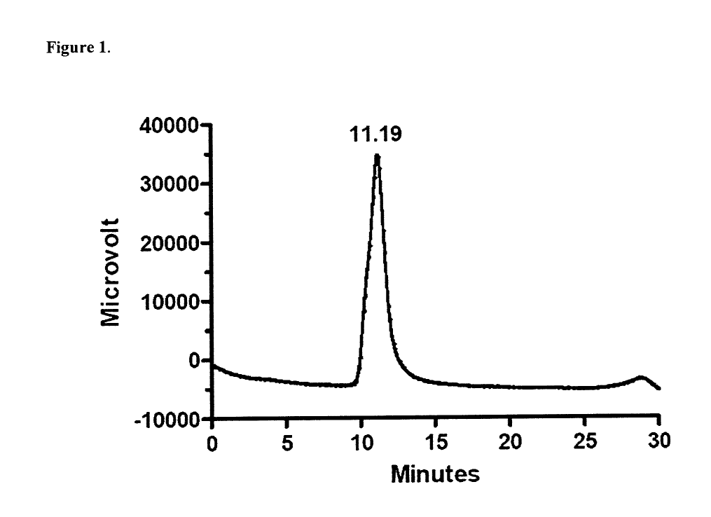

Additional set of five constructs are the following multimeric polypeptides (P21, P22, P27, P28, P29), containing peptides spanned by three alanine residues (AAA), were designed with Leto 1.2.3—the dedicated software for gene synthesis and optimized protein expression, and produced by known methods of synthetic gene synthesis. Cloning the optimized DNA sequence into pET30-a+ vector using the 5′ Nde 1-Bpu1102 I 3′ sites of the vector (as described in the DNA sequences below), subcloning to pET 30a+Vector to Nde I (ATG) without any tags. A double stop codon TAATAA followed by HindIII site and the Bpu1102 I site were also added to the gene at the 3′ end for sub-cloning to the vectors. Each of the DNA sequences start with the cloning restriction site sequence CAT for cloning into the expression vector. FIGS. 1-6 depict gel filtration analyses and/or gel electrophoresis results of the expressed P21, P22, P27, P28 and P29 polypeptides.

Polypeptide P21, 508 amino acids, contain the peptides of
SEQ ID NOS. 66-87
(SEQ ID NO: 122)
MNAIQESFTAAANDFEAEVAATMAAALNNAAALEDGQVLLVENTRYEDVDGAA
ADKAGKSLAPVAADLAAKLGQDVVFPGVTRGAELEAAINAAAKESKNDPELGKY
WASLGDGIFAAADAVAVEAEFAAAETQADSIEEIVEVVEGDNAAARLEKFLGGIE
DMPRIPDVMYVVDPHKEQIAVKEAKKLGIPVVAMVDTNAAADPDDIDVIIPANDD
AIRAVKLITAKLADAIIEGRQGAAAIRRNEELANSGAAALSRKDDEGQDGPIVDYIL
AAALDVTAKYQQAVRDIVALAAAAPGTDATIQALLPHLAAAQDGPIVDYILAAAV
HASKVLPKPAAFAAAGDKAELIEKAAAQLRVASKENNWNLPFADAAAVPVPTAA
ASAAITYFDSYRSADAAARKDKEGTFHYSWAAAESRGYTVAIYNRSKEKTEAAAV
IACHPEKNFVPSYDVESAAADVLEEISAKAPEDGKPCVTYIGPDGAAAESYDLMQH
LLGLSAEDMAEIFT.
The optimized DNA sequence of P21
(SEQ ID NO: 123)
CATAT GAATG CTATT CAAGA ATCTT TTACT GCTGC TGCTA ATGAT TTTGA
AGCTG AAGTT GCTGC AACTA TGGCT GCAGC ACTGA ACAAC GCCGC AGCAC
TGGAA GATGG TCAGG TACTG CTGGT CGAAA ACACC CGCTA CGAAG ACGTC
GACGG TGCTG CAGCA GATAA AGCAG GCAAA TCCCT GGCGC CAGTA GCAGC
TGACC TGGCA GCAAA ACTGG GCCAG GACGT GGTAT TCCCA GGTGT AACCC
GTGGT GCAGA ACTGG AAGCA GCGAT CAACG CAGCG GCGAA AGAAT CCAAA
AACGA TCCGG AACTG GGTAA ATACT GGGCC TCCCT GGGTG ACGGT ATTTT
CGCAG CTGCA GACGC AGTAG CTGTT GAAGC AGAAT TCGCT GCAGC GGAAA
CCCAG GCTGA CAGCA TCGAG GAAAT TGTGG AAGTC GTAGA AGGCG ACAAC
GCAGC TGCAC GTCTG GAGAA ATTCC TGGGT GGCAT CGAGG ATATG CCACG
CATCC CGGAT GTCAT GTACG TTGTG GACCC GCACA AAGAG CAGAT TGCGG
TCAAA GAGGC CAAAA AACTG GGTAT CCCGG TGGTG GCAAT GGTAG ATACC
AACGC AGCTG CGGAT CCAGA CGACA TCGAC GTCAT CATTC CGGCG AACGA
CGACG CTATC CGTGC AGTCA AACTG ATCAC GGCGA AACTG GCAGA CGCCA
TCATC GAAGG CCGTC AGGGT GCAGC AGCGA TTCGT CGTAA CGAGG AACTG
GCTAA CAGCG GTGCT GCTGC TCTGT CTCGT AAAGA CGACG AAGGT CAGGA
CGGTC CGATC GTCGA CTATA TTCTG GCAGC AGCAC TGGAC GTCAC CGCAA
AATAC CAGCA GGCTG TTCGT GACAT TGTTG CACTG GCTGC GGCAG CTCCA
GGTAC TGATG CGACC ATTCA GGCAC TGCTG CCACA TCTGG CTGCT GCACA
GGATG GTCCG ATCGT CGATT ACATT CTGGC TGCAG CGGTT CACGC AAGCA
AAGTC CTGCC GAAAC CGGCT GCTTT TGCAG CTGCT GGCGA CAAAG CTGAA
CTGAT CGAGA AAGCT GCTGC ACAGC TGCGC GTCGC GTCTA AAGAG AACAA
CTGGA ACCTG CCGTT CGCTG ACGCA GCTGC AGTTC CAGTT CCAAC TGCCG
CGGCT AGCGC TGCGA TTACG TATTT CGACT CCTAT CGTTC CGCAG ATGCA
GCAGC ACGCA AAGAT AAAGA GGGCA CGTTC CACTA TTCCT GGGCA GCGGC
TGAAT CTCGT GGCTA TACGG TCGCT ATCTA CAACC GCTCC AAAGA GAAAA
CGGAG GCAGC AGCAG TTATC GCGTG CCACC CGGAA AAAAA CTTCG TGCCG
TCTTA CGACG TCGAA AGCGC AGCTG CAGAT GTGCT GGAGG AGATC TCTGC
TAAAG CTCCG GAGGA CGGCA AACCG TGTGT GACTT ACATC GGTCC GGATG
GTGCA GCTGC TGAAA GCTAC GACCT GATGC AGCAT CTGCT GGGTC TGAGC
GCAGA GGATA TGGCC GAGAT CTTCA CGTAA TAAGC TTGCT GAGC.
Polypeptide P22, 492 amino acids, contain the peptides of SEQ
ID NOS. 89-102 (SEQ ID NO: 94 without the N-terminal Pro
residue) and SEQ ID NO: 27:
(SEQ ID NO: 124)
MKEKAMEVPAIKAAAKLFANYEANVKYQAIENAASHNGIFAALEAAAKWKVENS
WGDKVGTDGYFVASDAWMDEYTYQIVVRKELLTAEEQAAYGAEAAASFKYYVI
EVNPAAAGVHHNEIPELAAAARTDIQKIMVIGSGPIIIGQAAEFDYAGTQACAAASL
KEEGYEVVLVNSNPATIMTDKEIADKVYIEPITLEFVTRILRKEAAADALLPPTLGG
QTGLNMAMELSKNGILDELAAASAIDQAEDRDLFKQLMEELEQPIPESEIVNTVEE
AAAAQTMSDYENQMLRDASLSIIRALKIEGGCNVQLALDPAAATAKLNGFSDRKI
AELWGTTDDAAAVRQLRLENKIVPVYKMVDTCAAEFDSETPYFYSTYGWENESIA
AASDKESVLVLGSGPIRIGQGVEFDYATVHSVKAIQAAGYEAIAAAVLIPGYVIEVN
PRASRTVPFLSKVTNIAAAIIENLPKVMDKYGISSLAAAEELRQEVKE.
The optimized DNA sequence of P22 (1496 bp)
(SEQ ID NO: 125)
CATATGAAAGAAAAAGCTATGGAAGTTCCAGCTATTAAAGCTGCTGCAAAACTGTTTG
CTAATTATGAAGCCAACGTCAAATACCAGGCGATTGAAAACGCTGCGTCCCACAACGG
CATCTTTGCGGCGCTGGAGGCCGCTGCGAAATGGAAAGTTGAGAACTCTTGGGGTGAC
AAAGTGGGTACTGATGGCTACTTCGTAGCCAGCGATGCTTGGATGGACGAGTATACCT
ACCAGATTGTAGTGCGCAAAGAACTGCTGACCGCTGAGGAACAGGCAGCCTATGGTGC
GGAGGCGGCAGCATCCTICAAATACTATGTTATCGAAGTGAACCCGGCGGCTGCAGGT
GTGCATCACAACGAGATCCCGGAACTGGCGGCGGCGGCTCGTACCGATATCCAGAAAA
TCATGGTCATCGGTAGCGGTCCGATCATCATTGGTCAGGCGGCAGAATTTGACTACGC
AGGTACCCAGGCATGTGCTGCTGCCTCTCTGAAAGAAGAAGGCTACGAGGTAGTTCTG
GTAAACTCCAACCCGGCAACTATCATGACCGACAAAGAGATCGCGGACAAAGTGTAC
ATTGAACCGATCACCCTGGAATTCGTTACGCGTATTCTGCGTAAAGAAGCTGCGGCTG
ATGCGCTGCTGCCACCGACTCTGGGTGGCCAGACCGGTCTGAACATGGCTATGGAACT
GAGCAAAAACGGCATCCTGGATGAACTGGCGGCCGCTAGCGCTATCGATCAGGCGGA
AGATCGCGATCTGTTCAAACAGCTGATGGAGGAACTGGAGCAGCCGATCCCGGAATCC
GAAATTGTAAACACTGTGGAAGAAGCGGCGGCGGCTCAGACCATGTCCGACTACGAA
AACCAGATGCTGCGTGACGCCTCTCTGTCTATCATTCGTGCGCTGAAAATCGAAGGTG
GCTGCAACGTTCAGCTGGCTCTGGATCCGGCAGCAGCAACCGCGAAACTGAACGGCTT
CTCCGACCGCAAAATCGCTGAACTGTGGGGTACCACTGATGATGCTGCTGCTGTACGC
CAGCTGCGTCTGGAAAACAAAATCGTTCCGGTTTACAAAATGGTGGACACCTGCGCAG
CCGAATTCGACTCTGAAACTCCGTACTTCTATTCTACGTACGGTTGGGAAAACGAGTCT
ATCGCAGCCGCCTCTGACAAAGAATCTGTACTGGTTCTGGGCAGCGGCCCGATTCGCA
TCGGTCAGGGTGTTGAATTCGACTACGCGACCGTCCACTCCGTGAAAGCAATTCAGGC
AGCAGGCTATGAAGCCATCGCGGCAGCCGTGCTGATTCCAGGCTATGTTATCGAAGTT
AACCCGCGTGCCTCTCGTACCGTTCCGTTCCTGTCTAAAGTGACGAACATCGCCGCAGC
GATCATTGAAAACCTGCCGAAAGTTATGGACAAATACGGCATCAGCTCTCTGGCAGCG
GCGGAGGAACTGCGTCAGGAAGTCAAAGAGTAATAAGCTTGCTGAGC.
Polypeptide P27, 511 amino acids contain the peptides of SEQ
ID NOS. 26-31 and 38-49:
(SEQ ID NO: 126)
MKEKAMEVPAIKAAAVAYLVETEGASAGVMIAAAYYVDTITDVVRAEIGIDAAAS
HNPALDNGIKFFGGDGFKLDDEKEAEIEALLDAEEDTLPAAAIGLAFDGDSDRAAA
VDENGDIVDGDKIMYIIGKYLSAAATGKSLSELAAEVTIYPQKLVNIRVENAAAIIE
KMEEEMAGNGRILVRPSGTEPLLRVMAEAPTAAARDRLFKNVPEAAADNYYIKVP
AILDAAAETAAFATTLSKIVDMVELLGEVDTTGVAPTTTMADRKTVLRPDVAEEG
AAAMKITQEEVTHVANLSKLRFSEAAAHEKAVGDALDLSHALAAASIVTQVVESG
FKGIFLVAAASTKQHKKVILVGDGAVGSSYAFALVNQGIAQELGIEIPQAAASPKKI
YAAQYSDCADADLVVITAGAPQKPGETRLDLVGKNLAINAAAVLDGIETVISGAA
AYTGDQMILDGPHRGGDLRRARAGAANIVPNAAALRKVFEQESIDAAAVELLNLG
KEVIIVDNLSNSS.
Optimized DNA sequence of P27 (1561 bp):
(SEQ ID NO: 127)
CATATGAAAGAAAAAGCTATGGAAGTTCCAGCTATTAAAGCTGCTGCTGTTGC
TTACCTGGTTGAAACCGAAGGCGCTTCTGCTGGTGTGATGATTGCGGCAGCGTA
CTATGTAGACACTATCACCGACGTTGTCCGTGCGGAGATTGGCATTGATGCAGC
TGCTTCTCACAACCCAGCGCTGGATAACGGCATCAAATTTTTCGGCGGTGACGG
CTTCAAACTGGATGACGAGAAAGAAGCCGAGATTGAAGCTCTGCTGGACGCAG
AAGAGGATACGCTGCCGGCAGCCGCAATCGGTCTGGCTTTCGACGGCGATTCT
GATCGCGCAGCCGCAGTGGATGAGAACGGTGATATCGTAGACGGCGACAAAA
TTATGTACATCATCGGCAAATACCTGAGCGCTGCAGCGACCGGTAAATCCCTGT
CTGAACTGGCCGCTGAAGTCACGATTTACCCGCAGAAACTGGTTAACATCCGC
GTAGAAAACGCCGCCGCGATTATCGAAAAAATGGAGGAAGAGATGGCGGGTA
ACGGTCGTATCCTGGTTCGCCCGTCTGGCACTGAACCGCTGCTGCGCGTGATGG
CTGAAGCGCCAACTGCTGCGGCTCGTGACCGTCTGTTCAAAAACGTTCCGGAA
GCGGCAGCCGACAACTATTACATCAAAGTGCCAGCGATTCTGGATGCTGCCGC
TGAAACCGCTGCCTTCGCGACCACGCTGTCCAAAATCGTCGACATGGTCGAAC
TGCTGGGTGAAGTTGATACCACCGGTGTAGCCCCGACCACGACTATGGCGGAC
CGCAAAACTGTTCTGCGTCCGGACGTTGCTGAAGAGGGTGCTGCAGCCATGAA
AATCACCCAGGAAGAGGTGACTCATGTAGCGAACCTGTCTAAACTGCGTTTCA
GCGAAGCGGCTGCACACGAGAAAGCGGTTGGTGACGCACTGGACCTGTCTCAT
GCCCTGGCTGCTGCGAGCATCGTAACCCAGGTAGTTGAATCCGGTTTTAAAGGT
ATCTTTCTGGTGGCGGCAGCCTCTACTAAACAGCACAAAAAAGTGATCCTGGT
GGGTGACGGTGCTGTTGGCTCCTCTTATGCGTTCGCACTGGTGAACCAGGGTAT
TGCGCAGGAACTGGGTATCGAAATCCCACAGGCTGCAGCTTCCCCGAAAAAAA
TCTACGCTGCCCAGTACTCTGATTGCGCAGACGCGGATCTGGTAGTTATCACTG
CAGGTGCACCGCAGAAACCAGGTGAAACCCGCCTGGACCTGGTGGGCAAAAA
CCTGGCGATTAACGCAGCTGCAGTCCTGGATGGCACCGAAACCGTTATCTCTG
GTGCAGCTGCCTATACCGGTGATCAGATGATCCTGGATGGCCCGCACCGTGGT
GGTGATCTGCGTCGTGCTCGTGCAGGCGCTGCAAACATTGTTCCGAACGCTGCA
GCGCTGCGTAAAGTATTCGAACAGGAGTCCATCGACGCTGCAGCTGTTGAACT
GCTGAACCTGGGCAAAGAAGTGATCATCGTGGATAACCTGAGCAACAGCAGCT
AATAaAAGCTTGCTGAGC.
Polypeptide P28, 522 amino acids contain the peptides of
SEQ ID NOS. 50-65:
(SEQ ID NO: 128)
MTSAKEYIQSVFETVKAAALVLDRIEAITGIRPVFYELDVCDKAAAVMYYKNNIMS
TLALVEVMSEFNVKKIAAANPYGYTKVMLEQILKDVHVADSEWSIALLRAAADGS
FDIDPEIFELGIAAAMQFELIEPLNTLYKDEVRALGTELGMPDHIVWRQAAAQGVIC
VSEGANMPSAAALDAIKVYKENGIAAARNGHEAEFLQAVEEFFNTLEPVFEKHPE
YIEENILARITEPERAAAMRFCQSFMTELQKHIGPSLDVPAGDIGVGGREIGYLYGQ
YKRLNQFDAAAGVLTGKPLGFGGSLIRPEATGYGLAAAYYTEEMLKANGNSFAG
KKVVISGSGNVAQYALQKATELGATVISAAASDSNGYVIDENGIDFDLLVDVKEK
RRARLTEYAAEKATATYHAAAYAGNYDIALPCATQNEINGEAAKRLVAAAYGPA
KAANAGGVAVSALEMSQNSLRLSWTREEVDAAARLKDIMTNIFNTAKTTSETYGL
DKDYLAGANIAAFENVANAMIAQG.
Optimized DNA sequence of P28 (1588 bp):
(SEQ ID NO: 129)
CATATGACTTCTGCTAAAGAATATATTCAGTCTGTTTTTGAAACTGTTAAAGCAGCTGC
TCTGGTGCTGGATCGTATCGAAGCTATCACGGGCATTCGCCCGGTGTTCTATGAACTGG
ATGTTTGCGACAAAGCGGCAGCAGTGATGTACTATAAAAACAACATCATGTCCACCCT
GGCGCTGGTCGAAGTGATGTCTGAATTCAACGTTAAAAAAATTGCTGCTGCGAACCCG
TACGGCTACACGAAAGTGATGCTGGAACAGATCCTGAAAGATGTGCACGTTGCGGATT
CCGAATGGTCTATTGCACTGCTGCGTGCGGCTGCGGACGGCAGCTTCGACATTGACCC
GGAAATTTTCGAACTGGGTATCGCGGCAGCAATGCAGTTCGAACTGATCGAACCGCTG
AACACCCTGTATAAAGATGAAGTTCGTGCACTGGGCACCGAGCTGGGCATGCCGGACC
ACATCGTTTGGCGCCAGGCCGCCGCTCAGGGTGTTATCTGTGTTAGCGAGGGTGCAAA
CATGCCATCCGCGGCCGCACTGGACGCAATCAAAGTATATAAAGAGAACGGCATCGCG
GCTGCACGTAACGGCCACGAGGCGGAATTCCTGCAGGCGGTAGAAGAATTTTTCAACA
CCCTGGAACCGGTGTTTGAGAAACACCCGGAATACATTGAAGAAAACATTCTGGCCCG
CATCACCGAACCGGAACGCGCGGCTGCGATGCGTTTCTGCCAGTCTTTCATGACTGAA
CTGCAGAAACATATCGGTCCGTCCCTGGACGTTCCAGCGGGTGATATTGGCGTAGGTG
GTCGTGAAATTGGCTACCTGTACGGCCAGTACAAACGTCTGAACCAGTTTGACGCGGC
TGCTGGTGTGCTGACGGGCAAACCGCTGGGTTTCGGTGGTAGCCTGATCCGTCCAGAG
GCTACTGGTTACGGTCTGGCGGCTGCCTACTACACCGAGGAAATGCTGAAAGCAAACG
GCAACAGCTTCGCTGGCAAAAAAGTTGTAATCTCTGGTAGCGGTAACGTCGCACAGTA
CGCTCTGCAGAAAGCTACCGAGCTGGGTGCTACCGTAATCTCCGCTGCAGCCTCCGATT
CTAACGGTTATGTGATCGACGAAAACGGTATCGACTTTGACCTGCTGGTTGACGTAAA
AGAAAAACGTCGTGCGCGCCTGACCGAATATGCGGCCGAGAAAGCAACTGCTACCTAT
CATGCCGCGGCCTACGCGGGCAACTACGACATCGCACTGCCATGTGCGACTCAGAACG
AAATCAACGGCGAAGCGGCGAAACGTCTGGTCGCGGCTGCGTACGGTCCAGCAAAAG
CTGCTAACGCCGGTGGTGTTGCCGTATCTGCCCTGGAAATGTCTCAGAACTCTCTGCGC
CTGAGCTGGACCCGTGAAGAGGTCGATGCGGCCGCACGTCTGAAAGATATCATGACCA
ACATCTTCAACACCGCGAAAACTACGTCTGAGACCTACGGTCTGGATAAAGATTACCT
GGCTGGCGCAAACATCGCGGCATTCGAAAACGTTGCTAACGCCATGATTGCACAGGGC
TAATAaAAGCTTGCTGAGC.
Polypeptide P29, 522 amino acids contain the peptides of SEQ
ID NOS. 103-121:
(SEQ ID NO: 130)
MTTNRLQVSLPGLDLKNPIIPASGCFGFGQEYAKYYDLAAAEAALEMYLAGASAI
GVGTANFTNPYACPAAAQMDGAILVVASTAAAQERILEDINLQVTSGEVVSAAAI
VSEIKLDWSEDEDKEVQKIAYKRQILAELGLDAAALLKEFQELETFADFAAAKLAE
FELAGKPAAALSGVPEAKDKAAAYKEWGMPAHAAAFLPFGVSPDFMDARINVLW
ASAPSTILPDTTYYAEEHPQREELAAATLWKESSANLLKAYDFSDEEIEDLLEKRLE
LDRRVAAAWSLLKATLILSVVNLSTSYLTEDIRVLAAAYQRVYRYLDKRVDAAAL
KDSRTAKYHKRLQIVLFAAAILDACPLVLDCRVDRIVEEDGICHIFAKILERLVAPE
AAALDEKGHFKNQLFAPTYFMGDGAAAMNIIEEIMTKLREDIRNIAIIAAAVDELL
KQSETLDARTELAERAMDSNDIEKERGITIAAAEVVDEVLELFIELGADDDQLDFP
VVYASAINGTSSLSDDPADQE.
Optimized DNA sequence of P29 (1588 bp):
(SEQ ID NO: 131)
CATATGACTACTAACCGTCTGCAAGTATCTCTGCCAGGTCTGGATCTGAAAAACCCGAT
TATTCCGGCATCCGGCTGCTTTGGCTTTGGTCAGGAGTACGCCAAATACTATGACCTGG
CTGCAGCCGAAGCGGCACTGGAAATGTATCTGGCTGGTGCTAGCGCAATTGGCGTAGG
TACGGCTAACTTTACGAACCCGTACGCCTGTCCAGCTGCAGCCCAGATGGATGGCGCA
ATCCTGGTAGTGGCTTCCACTGCTGCCGCTCAGGAACGCATTCTGGAAGACATCAACCT
GCAGGTGACTTCTGGCGAGGTAGTGTCTGCTGCAGCGATCGTCTCTGAGATCAAACTG
GATTGGAGCGAGGACGAGGACAAAGAAGTGCAGAAAATCGCATACAAACGTCAGATC
CTGGCTGAACTGGGTCTGGATGCTGCAGCGCTGCTGAAAGAGTTCCAGGAGCTGGAAA
CCTTTGCGGACTTTGCTGCAGCCAAACTGGCAGAATTCGAACTGGCTGGTAAACCGGC
AGCGGCACTGTCTGGCGTGCCAGAAGCAAAAGACAAAGCGGCAGCTTACAAAGAGTG
GGGTATGCCGGCACACGCAGCCGCTTTTCTGCCGTTTGGTGTGTCTCCGGACTTCATGG
ACGCACGTATCAACGTTCTGTGGGCCTCTGCTCCATCTACTATCCTGCCGGATACCACG
TACTATGCGGAGGAACACCCACAGCGTGAGGAACTGGCAGCTGCAACCCTGTGGAAA
GAATCCAGCGCAAACCTGCTGAAAGCGTACGATTTCAGCGACGAAGAGATCGAGGAT
CTGCTGGAGAAACGCCTGGAACTGGACCGTCGCGTAGCTGCAGCTTGGAGCCTGCTGA
AAGCCACTCTGATCCTGAGCGTAGTCAACCTGAGCACGAGCTATCTGACGGAAGACAT
TCGCGTTCTGGCAGCTGCCTATCAGCGTGTATACCGCTATCTGGACAAACGCGTTGACG
CAGCTGCCCTGAAAGATTCTCGCACTGCGAAATACCACAAACGTCTGCAGATCGTCCT
GTTCGCTGCCGCAATTCTGGACGCTTGTCCGCTGGTCCTGGATTGTCGTGTAGACCGTA
TCGTAGAGGAAGACGGTATCTGTCACATCTTCGCCAAAATTCTGGAACGCCTGGTAGC
TCCGGAAGCTGCAGCTCTGGACGAGAAAGGTCACTTCAAAAACCAGCTGTTCGCACCA
ACCTACTTCATGGGTGACGGTGCAGCCGCAATGAACATTATCGAGGAGATCATGACGA
AACTGCGTGAGGACATCCGCAACATTGCAATCATCGCTGCAGCGGTAGACGAACTGCT
GAAACAGAGCGAAACTCTGGACGCTCGTACTGAACTGGCAGAGCGTGCAATGGACTCC
AACGATATCGAGAAAGAGCGTGGTATCACTATCGCTGCGGCAGAAGTTGTAGACGAG
GTCCTGGAGCTGTTTATCGAGCTGGGTGCAGACGATGACCAGCTGGATTTCCCGGTGG
TCTATGCTTCCGCTATCAACGGCACCTCCTCTCTGAGCGATGACCCAGCTGATCAGGAG
TAATAAAAGCTTGCTGAGC.

Following expression in, for example, E. coli and purification, the resulting multimeric polypeptides are tested for their vaccine potential in the intranasal and intraperitoneal mouse challenge models.

### Example 4

### Detailed Protocol for Expression, Refolding and Purification of P21

Expression—A DNA optimized clone denoted P21 was used. For amplification, XL-1 cells were transformed with the pET30a+plasmid encoding the polypeptide. Subsequently DNA from two clones was prepared with a QIAGEN kit and used for transformation of BL-21 Codon and pLys cells. Four clones of each were picked, propagated and stored as glycerol cultures. Four clones in BL-21 codon cells were grown in 30 ml TB medium at 37° C. until the A₅₉₅=0.9, and then expression was induced with 0.4 mM IPTG. After 4 hours the cells were spun and frozen. Next day each pellet was suspended in 5 ml cold DDW containing 10 mM EDTA and 10 mM Tris-HCl buffer pH 8.0, shaken for 30 mM, sonicated and centrifuged. The supernatant was kept on ice, and the pellet was sonicated and centrifuged again. The soluble (cytosolic) and insoluble inclusion bodies (IBs) fractions were evaluated for mini-expression using 12% SDS-PAGE. The expression of P21 was detected only in the insoluble fraction in both induced and non-induced Codon cells. The expression in pLys cells was also good but not as high as in Codon cells.

Preparation of inclusion bodies (IBs) extract—In view of preliminary results along with the fact that no expression was detected in the soluble fraction of cell lysate, it was concluded that the insoluble protein found after one cycle of sonication of the IBs is slowly solubilized by the following washing cycles and finally lost. To verify this hypothesis, a new transformation of the plasmid to Codon cells was performed and 10 more clones were tested for mini expression 4 and 24 hr after induction with IPTG, all showing the same feature. The P21 polypeptide could be identified in the washout fractions.

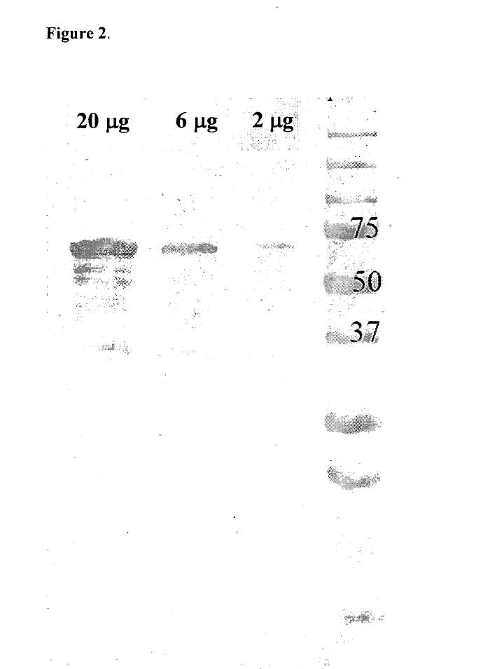

Clone no. 4 was chosen for this large-scale preparation. After sonication and precipitation, the precipitate was suspended in UPW, sonicated and centrifuged. This procedure was repeated 3 more times, yielding respectively 4 supernatant fractions termed S1, S2, S3 and S4. SDS-PAGE analysis revealed that most of P21 was gradually solubilized by UPW; however, S1 and S2 still contained many accompanying proteins. Therefore to achieve better purity, even at the expense of lower yield, only S3 and S4 were used for further purification. Those fractions (200 ml) were combined, precipitated with AmSO₄ to 15% saturation, and centrifuged. The precipitate was dissolved in 200 ml of 10 mM Tris-HCl buffer pH 8 containing 0.1 mg PMSF and dialyzed overnight against the same buffer. Next morning the dialysate was applied onto a Q-Sepharose column above. The flow-through fraction was collected, concentrated to 60 ml and applied onto Superdex 200 preparative column, pre-equilibrated and developed with TN buffer pH 8.0, containing 0.1 mg PMSF per ml in 3 consecutive applications. The protein appeared as a single peak corresponding to molecular mass of an oligomer. Five ml samples were collected and analyzed for P21 content by SDS-PAGE. The left shoulder of the peak contained the main band of ˜65 kDa and a lower molecular mass band of ˜34 kDa. The latter gradually disappeared in the right shoulder of the peak. Subsequently fractions from the two other separations corresponding to tubes 6-9 were pooled, concentrated to 35 ml, and desalted by gel-filtration on a G-25 Sephadex column (bed volume 480 ml) pre-equilibrated with 0.1% NaHCO₃. Then the eluted P21 was immediately filter-sterilized and lyophilized in 0.25 mg/vial. The protein concentration was calculated the A₂₈₀, assuming that absorbance of 0.67=1 mg/ml as calculated by the DNAman program. The overall yield was 70 vials of 0.25 mg each. The lyophilized P719 could be easily dissolved in UPW. Its gel-filtration profile is presented in FIG. 1, showing that P21 appears as a monomer under non-denaturing conditions. In order to quantify the purity of the purified P21, a lyophilized sample was analyzed by SDS-PAGE in the presence of ME at 3 concentrations (20, 6 and 2 μg per lane), scanned and quantified (FIG. 2). The main ˜65 kDa was of larger apparent molecular size than the theoretical value of 52818 Da. As estimated by densitometry, the sample run at 6 μg per lane consisted of ˜42% of the ˜65 kDa protein and the band seen at ˜34 kDa was ˜11%.

### Example 5

### Detailed Protocol for Expression, Refolding and Purification of P22

Expression—DNA clone P22 was used. In order to amplify the DNA, XL-1 cells were transformed with the pET30 plasmid encoding this protein. Subsequently DNA from 2 clones was prepared with QIAGEN kit and used for transformation of BL-21 Codon and pLys cells. Four clones of each were picked up, propagated and stored as glycerol cultures. Four clones in BL-21 codon cells were grown in 30 ml TB medium at 37° C. till the OD₅₉₅ reached 0.9 and then expression was induced with 0.4 mM IPTG. After four hours the cells were spun and frozen. Next day each pellet was suspended in 5 ml cold DDW containing 10 mM EDTA and 10 mM Tris-HCl buffer pH 8.0, shaken for 30 min, sonicated and centrifuged. The supernatant was kept on ice and the pellet was sonicated and centrifuged again. The soluble (cytosolic) and insoluble inclusion bodies (IBs) fractions were evaluated for mini-expression using 12% SDS-PAGE. The expression of P22 was detected only in the insoluble fraction in both induced and non-induced bacteria but not in the supernatant.

Large-scale fermentation—Clone no 4 was used for large-scale preparation. Fermentation of 2.5 l (5×500 ml flasks) was carried out at 37° C. in TB medium. After three hours when the cells reached OD₅₉₅=0.9, 0.4 mM of IPTG was added, the fermentation was continued for four hours, and then the cells were centrifuged and stored in −20° C. The cell pellet was suspended in 600 ml UPW containing 10 mM EDTA, 10 mM Tris-HCl buffer pH 8.0, DNAse (10 μg/ml) and lysosyme (0.2 mg/ml) for 3 h in cold. Then the suspension was sonicated, centrifuged and IBs were fully purified as described for P21, suspended in 100 ml UPW, divided into 3×33 ml aliquots and frozen. Aliquot were analyzed by SDS-PAGE. Semi-quantitative evaluation based on P710 run as a positive control shows that 5 μl of the suspension contains ˜10 μg P22, meaning that there are ˜200 mg in the entire stored suspension. A low molecular protein of unknown nature (˜13 kDa) was co-expressed with P22.

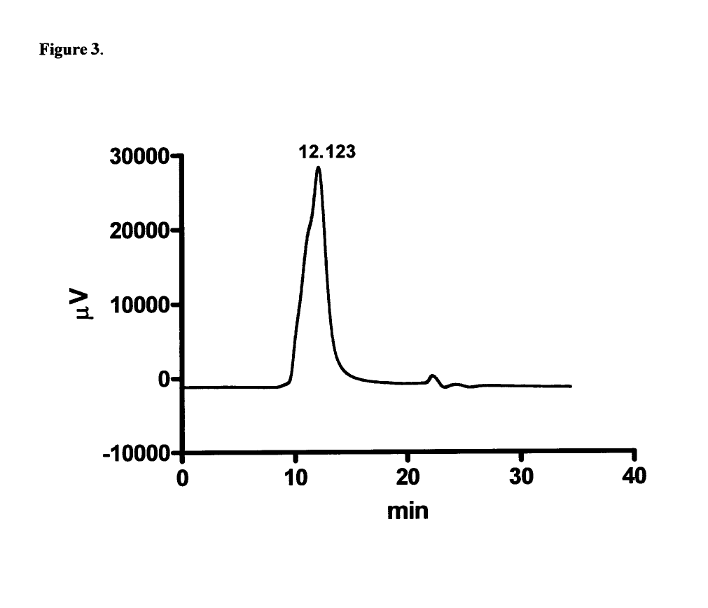

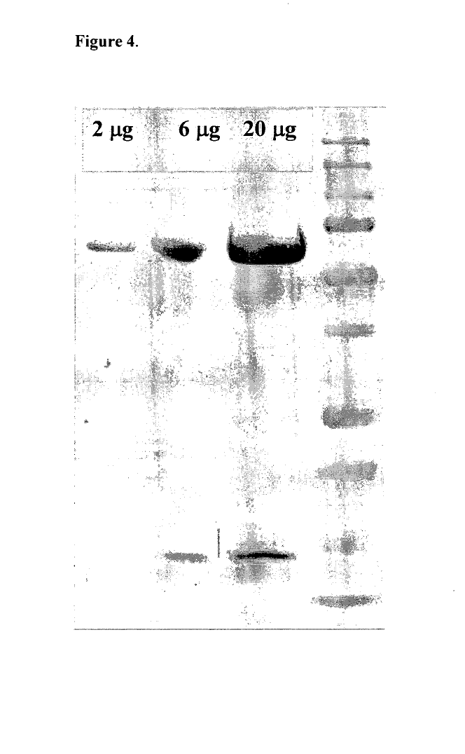

Large scale preparation—24 ml IBs (from 2.5 L of fermentation culture which were suspended in 100 ml UPW) were refolded in a final volume of 100 ml of 4.5 M urea, 40 mM Tris Base, 1 mM cysteine and 0.1 mM PMSF, adjusted to pH 11.3. After 2 hr at 4° C., 200 ml of cold UPW was added and the solution was stirred for 1 hr. at 4° C. The refold was then dialyzed against 10 mM Tris-HCl buffer pH 8.0 and 0.1 mM PMSF for 2-3 days with 2 exchanges per a day. The 300 ml refolding solution was centrifuged and adjusted to 0.3 M NaCl, applied on Q-Sepharose pre-equilibrated with 10 mM Tris-HCl buffer pH 8.0, 0.3 M NaCl and 0.1 mM PMSF. The breakthrough material was concentrated to ˜60 ml which were subsequently separated in 3×20 ml portions on a preparative 200 Superdex column pre-equilibrated with TN buffer at pH 8.0 and 0.1 mM PMSF at 4° C. The column was developed at 2 ml/min. Sixty minutes after application, 14 tubes containing 5 ml samples were collected. The samples were put at 4° C. immediately and stored at −20° C. Aliquot from almost every tube were thawed and analyzed by SDS-PAGE. The experiment was repeated in an identical manner. Gel filtration removes almost all proteins of lower molecular mass, however the main expected band of ˜55-60 kDa is accompanied by a small band of ˜15 kDa. This band is less visible most likely because the amount of the applied protein was small but this band does not result from reduction of S—S bonds. Aliquots from those tubes were also analyzed by gel filtration on an analytical Superdex 75 column equilibrated with TN pH 8.0. Tubes 2-5 contained dimeric P22, and tubes 6-14 contained monomeric forms of P22. All frozen samples from the six preparative Superdex columns preparations that included both monomeric and dimeric fractions were thawed and pooled (340 ml with OD₂₈₀=0.2) yielding ˜68 mg, assuming the theoretical specific absorbance being 1.00 for 1 mg/ml. Those fractions were concentrated to ˜40 ml and desalted on G-25 Sephadex (2.6×100 cm) pre-equilibrated with NaHCO₃ (1 mg/ml). The eluate was adjusted with UPW to 0.5 mg/ml, distributed into vials at 0.25 mg aliquot/vial, and lyophilized. A total of 218 tubes (59 mg) were obtained and stored at −20° C. Each vial contains 0.20 mg of NaHCO₃. The lyophilized P22 could be easily dissolved in UPW. Its gel-filtration profile is presented in FIG. 3. MS analysis of polypeptide composition revealed only 6% successful identification to unrelated proteins. Three attempts to identify the molecular mass by MS analysis failed for an unknown reason. In order to verify quantitatively the purity of the purified P22, a lyophilized sample was analyzed by SDS-PAGE in the presence of ME at 3 concentrations (20, 6 and 2 μg per lane), scanned and quantified (FIG. 4). The main band of ˜55 kDa molecular as estimated by densitometry of the sample run at 20 μg per lane consisted of 75% of the signal, and the lower bands of ˜40, 35, 25 and 14 kDa consisted respectively of 4, 5, 2 and 14%. The purified P22 preparation (0.2 mg/ml) contained 100 EU/mg or 10 ng/mg endotoxin.

### Example 6

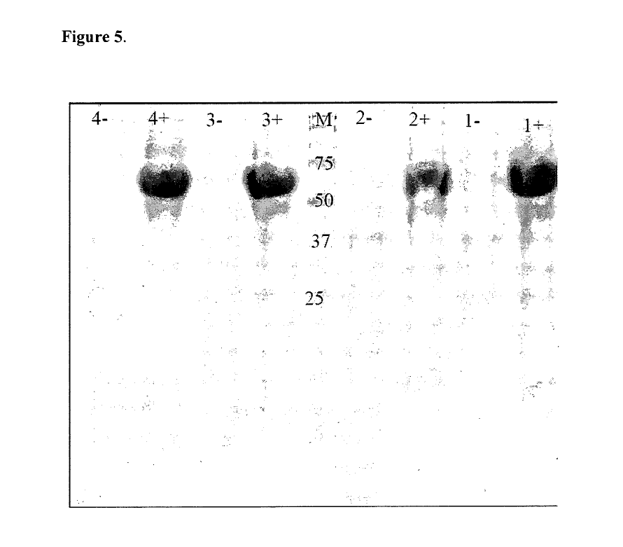

A DNA clone labeled P29 was used. In order to amplify the DNA, XL-1 cells were transformed with the pET30 plasmid encoding this protein. Subsequently DNA from 2 clones was prepared with a QIAGEN kit and used for transformation of BL-21 Codon and pLys cells. Four clones of each were picked, propagated and stored as glycerol cultures. Four clones in BL-21 codon cells were grown in 30 ml TB medium at 37° C. until OD₅₉₅=0.9, and then expression was induced with 0.4 mM IPTG. After 4 hours the cells were spun and frozen. Next day each pellet was suspended in 5 ml cold DDW containing 10 mM EDTA and 10 mM Tris-HCl buffer pH 8.0, shaken for 30 mM, sonicated and centrifuged. The supernatant was kept on ice, and the pellet was sonicated and centrifuged again. The soluble (cytosolic) and insoluble inclusion bodies (IBs) fractions were evaluated for mini-expression using 12% SDS-PAGE. Very good expression of P29 was detected in the insoluble fraction (FIG. 5) in induced pLys cells but not in the supernatant.

### Example 7

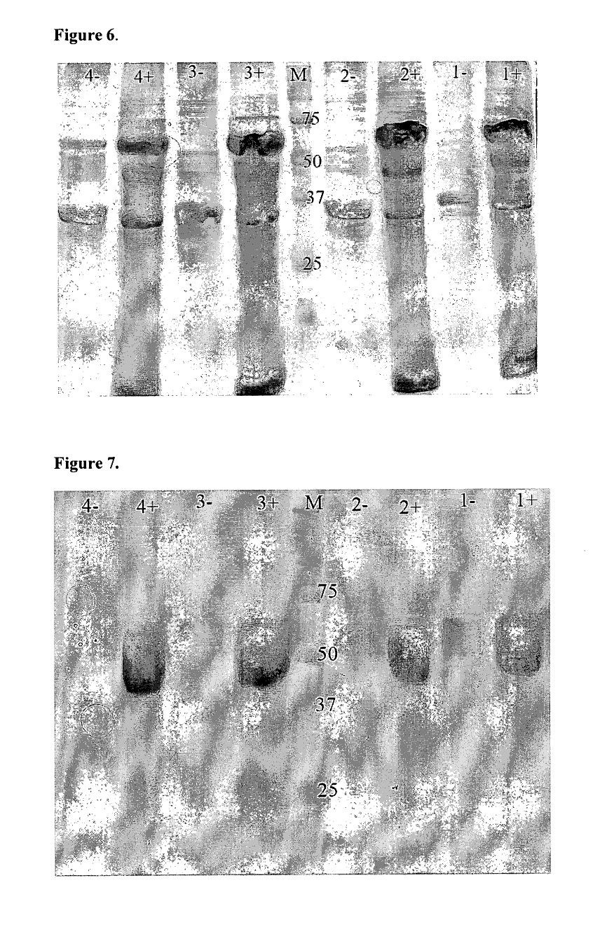

DNA clone P27 and 28 were used. In order to amplify the DNA, XL-1 cells were transformed with the pET30 plasmid encoding this protein. Subsequently DNA from 2 clones was prepared with QIAGEN kit and used for transformation of BL-21 Codon and pLys cells. Four clones of each were picked up, propagated and stored as glycerol cultures. Four clones in pLys cells were grown in 30 ml TB medium at 37° C. till the OD₅₉₅ reached 0.9 and then expression was induced with 0.4 mM IPTG. After 4 hours the cells were spun and frozen. Next day each pellet was suspended in 5 ml cold DDW containing 10 mM EDTA and 10 mM Tris-HCl buffer pH 8.0, shaken for 30 min, sonicated and centrifuged. The supernatant was kept on ice and the pellet was sonicated and centrifuged again. The soluble (cytosolic) and insoluble inclusion bodies (IBs) fractions were evaluated for mini-expression using 12% SDS-PAGE (FIGS. 6 and 7). The expression of P27 and P28 were detected only in the insoluble fraction of induced pLys bacteria but not in the supernatant.

### Example 8

### Testing the Efficacy of the Peptides and Peptide-Multimers

Immunogenic peptides are synthesized, or produced recombinantly, and used individually, as peptide-multimers, conjugated to polysaccharides or in different combinations as part of fusion polypeptides with or without a carrier or adjuvant sequence. The peptide compositions are tested, with or without an external adjuvant for their vaccine potential in several in-vitro, by neutralization of the bacteria ex-vivo and in-vivo models. Cross protection against capsularly and genetically unrelated bacterial strains is also tested. In certain cases, antibodies produces against selected peptides and polypeptides are used. The following models are used to test the efficacy:
i. In vitro model in which interference of bacterial adhesion to cultured upper and lower respiratory tract epithelial cells and to endothelial cells by the peptides, chimeric polypeptides and antisera against them;
ii. To evaluate the stage at which the immune system prevents disease two in vivo tests are used: in vaccinated mice the extent of nasopharyngeal, lung blood and spleen colonization is determined following challenge with S. pneumoniae tagged with luciferase is monitored using the bioluminescence imaging using an IVIS imaging system;
iii. Ex-vivo neutralization with antiserum against the peptides—Several hundreds CFU of S. pneumoniae strain 3 (WU2) are ex-vivo neutralized with mouse or rabbit diluted serums antiserum against the peptides and polypeptides for 1 hr and used to challenge 7 week old BALB/c or CBA/Nxid mice intraperitonealy. Negative control mice are challenged with S. pneumoniae strain 3 (WU2) after neutralization with negative control sera obtained from adjuvant alone injected animals. Positive control mice are challenged with S. pneumoniae strain 3 (WU2) after neutralization with mouse or rabbit anti Non-lectins serum. Survival is monitored for seven days.
iv. Mouse model for systemic infection—For systemic S. pneumoniae lethal challenge-mice immunized with a peptide/polypeptide formulated with adjuvant (i.p.) or intravenous (i.v.) or SC, are challenged with a lethal dose of S. pneumoniae serotype 3 strain WU2. The inoculum's size is determined to be the lowest that cause 100% mortality in the control mice within 96-120 hours. Survival is monitored daily. Immunization of mice with the adjuvant alone serves as negative control and with bacterial cell-wall non lectin fraction with the adjuvant serves as positive control
v. Mouse models for upper respiratory lethal infections—Mice immunized with peptide/polypeptide in adjuvant, with adjuvant alone as negative control and with non lectin as positive control, are inoculated intranasally with a lethal dose of S. pneumoniae serotype 3 strain WU2. Survival is monitored daily.
vi. Mouse models for upper respiratory S. pneumoniae colonization—mice immunized with peptide/polypeptide in adjuvant, with adjuvant alone as negative control and NL as positive control are anaesthetized with isoflurane, and inoculated intranasally with a sublethal dose of S. pneumoniae serotype 3 strain WU2 (in 25 μl PBS). The nasopharynx, and the lungs are excised homogenized and plated onto blood agar plates for bacteria enumeration;
vii. Otitis media models. Otitis media models in chinchilla and the rat (developed for example according to Chiavolini et al., 2008, Clinical Microbiology Reviews, 21:666-685; Giebink, G. S. 1999, Microb. Drug Resist., 5:57-72; Hermansson et al., 1988, Am. J. Otolaryngol. 9:97-101; and Ryan et al., 2006, Brain Res. 1091:3-8), are utilized to test the effectiveness of peptides and multimers according to the invention. The ability of the peptides and multimers to protect those animals from developing otitis media following intranasal challenge is studied.

### Example 9

### In Vivo Models for Testing the Multimeric Polypeptides P21, P22, P27, P28 and P29

### Vaccine Potential in the Intranasal Lethal Challenge Mouse Model

CBA/Nxid mice are immunized be subcutaneously (SC) with 10 or 20 microgram of multimeric polypeptide or controls emulsified either with CFA/IFA/IFA. The first immunization is performed with CFA while the booster immunization is performed at days 7 and 21 with IFA. Non-lectin proteins fraction of S. pneumonia serotype 3 (WU2) cell wall proteins serves as a positive control and PBS as negative controls. Mice are challenged intranasally at day 28 with 5×10⁵CFU lethal dose of S. pneumoniae serotype 3 strain WU2. The survival will is monitored daily over the next seven days and the experiment is terminated at this point.

### Vaccine Potential in Nasopharyngeal and Lung Bacterial Colonization Mouse Model

CBA/Nxid mice are subcutaneously (SC) or intramuscularly (IM) immunized with protein or controls emulsified either with CFA/IFA/IFA or CCS/C®, respectively. Ten or 20 microgram of multimeric polypeptide emulsified in CFA are used for immunization at day 0 and in the subsequent immunization at day 14 and 28 P21 or controls are emulsified in IFA. 3, 10 and 20 microgram of P21 are emulsified CCS/C® at antigen:adjuvant ratio of 1:100 and 3 microgram P21 are emulsified with CCS/C® at antigen:adjuvant ratio of 1:200. Non-lectin proteins fraction of S. pneumonia serotype 3 ('WU2) cell wall proteins serves as a positive control and PBS as negative controls. The immunization with CCS/C® is performed on days 0, 14, and 28. Mice are challenged at day 42 intranasally with a 1.25×10⁵CFU sub-lethal dose of S. pneumoniae serotype 3 strain WU2. Three and 48 hours after the challenge the mice are euthanized and the nasopharynx and the left lung are excised homogenized and plated in serial dilutions onto blood agar plates for enumeration.

### Vaccine Potential in the Intraperitoneal Lethal Challenge Mouse Model

CBA/Nxid mice are subcutaneously (SC) or intramuscularly (IM) immunized with multimeric polypeptide or controls emulsified either with CFA/IFA/IFA or CCS/C®, respectively. Non-lectin proteins fraction of S. pneumonia serotype 3 (WU2) cell wall proteins serves as a positive control and PBS as negative controls. The amounts of antigens and the experimental schedules are as described above. Mice are challenged intraperitoneally with 100 CFU lethal dose of S. pneumoniae serotype 3 strain WU2. The survival is monitored daily over the next seven days and the experiment is terminated at this point.

### Vaccine Potential in the Ex Vivo Bacterial Neutralization in the Intraperitoneal Lethal Mouse Model

S. pneumoniae serotype 3 strain WU2 or serotype 2 strain D39 are incubated for one hour with preimmune sera or sera obtained from mice immunized with the multimeric polypeptide as described above, non-lectin fraction or adjuvant only at 1:10 dilution. The mice are inoculated intraperitoneally with a 200 CFU lethal dose of bacteria. Survival is monitored daily over the next seven days when the experiment is terminated.

### Vaccine Potential of Multimeric Polypeptides: Profile of the Protective Immune Response

BALB/c or CBA/Nxid mice will be subcutaneously (SC) or intramuscularly (IM) immunized with P21 and P22 or controls emulsified either with CFA/IFA/IFA or CCS/C®, respectively. Non-lectin proteins fraction of S. pneumonia serotype 3 (WU2) cell wall proteins serves as a positive control and PBS as negative controls using the experimental regimens and schedules described above In experiments 1 and 2. CD4 T cells are harvested from the lymph nodes or the spleen using anti CD4 antibodies bound to magnetic beads. These CD4 T cells are then co-culture with dendritic cell prepared 8 days prior to the CD4 T cell harvest prepared from naïve mice. At predetermined time intervals the supernatant is collected and cytokine types and level of expression is determination in multi cytokine detection kit assays and the cell are lysed with a chaotic buffer for cytokine mRNA level determination by real time PCR assays.

### Vaccine Potential of Multimeric Polypeptides in Opsonophagocytosis Analysis

Alveolar macrophages or bone marrow derived macrophages will be harvested and incubated with S. pneumoniae strain R6 pretreated with sera obtained from shame or protein immunized mice (from the above described experiments). Following 1 hours incubation the cells will be treated with antibiotic for 30 minutes the cell will be lyzed and plated onto blood agar plates for enumeration. Alternatively, the bacteria will be labeled with carboxyfluorescein diacetate (CFDA) and nuclear staining is performed using Hoechst 33342 and the analysis is done using either flow cytometry or confocal microscopy. Quantification is done by counting the total number of cells and the number of cells that phagocytosed bacteria.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1.-40. (canceled)

41. A synthetic or recombinant peptide of 9-50 amino acids derived from the sequence of a Streptococcus pneumoniae (S. pneumoniae) cell wall or cell membrane protein associated with an age-dependent immune response, and variants and analogs thereof, having at least about 90% identity to said synthetic or recombinant peptide, wherein the S. pneumoniae cell wall or cell membrane protein is selected from the group consisting of SEQ ID NO:1 to SEQ ID NO:25 and the synthetic or recombinant peptide is selected from the group consisting of any one of SEQ ID NO:26-121 and variants and analogs thereof:

| | SEQ |
|---|---|
| | ID |
| | NO: |
| IIENLPKVMDKYGISSL | 101 |
| AVAYLVETEGASAGVMI | 26 |
| MKEKAMEVPAIK | 27 |
| YYVDTITDVVRAEIGID | 28 |
| ASHNPALDNGIKFFGGDGFKLDDEKEAEIEALLDAEEDTLP | 29 |
| IGLAFDGDSDRLIAVDENGDIVDGDKIMYIIGKYLS | 30 |
| TGKSLSELAAEVTIYPQKLVNIRVEN | 31 |
| IIEKMEEEMAGNGRILVRPSGTEPLLRVMAEAPT | 32 |
| TAQWNNHRV | 33 |
| LDIPAIKGINPDTD | 34 |
| GSYHDVDSSETAFKIAASLI | 35 |
| HKIGETHEGASQMDWMEQEQE | 36 |
| LMMKYLEGEEITNEELKAGIRKATINVEFFPVLC | 37 |
| RDRLFKNVPE | 38 |
| DNYYIKVPAILD | 39 |
| ETAAFATTLSKIVDMVELLGEVDTTGVAPTTTMADRKTVLRPDVA | 40 |
| EEG | |
| MKITQEEVTHVANLSKLRFSE | 41 |
| HEKAVGDALDLSHAL | 42 |
| SIVTQVVESGFKGIFLVA | 43 |
| STKQHKKVILVGDGAVGSSYAFALVNQGIAQELGIIEIPQ | 44 |
| SPKKIYAAQYSDCADADLVVITAGAPQKPGETRLDLVGKNLAIN | 45 |
| VLDGTETVISG | 46 |
| YTGDQMILDGPHRGGDLRRARAGAANIVPN | 47 |
| ALRKVFEQESIDAA | 48 |
| VELLNLGKEVIIVDNLSNSS | 49 |
| LVLDRIEAITGIRPVFYELDVCDK | 50 |
| VMYYKNNIMSTLALVEVMSEFNVKKI | 51 |
| NPYGYTKVMLEQILKDVHVADSEWSIALLR | 52 |
| DGSFDIDPEIFELGI | 53 |
| MQFELIEPLNTLYKDEVRALGTELGMPDHIVWRQ | 54 |
| MTSAKEYIQSVFETVK | 55 |
| QGVICVSEGANMPS | 56 |
| LDAIKVYKENGI | 57 |
| RNGHEAEFLQAVEEFFNTLEPVFEKHPEYIEENILARITEPER | 58 |
| MRFCQSFMTELQKHIGPSLDVPAGDIGVGGREIGYLYGQYKRLN | 59 |
| QFD | |
| GVLTGKPLGFGGSLIRPEATGYGL | 60 |
| YYTEEMLKANGNSFAGKKVVISGSGNVAQYALQKATELGATVIS | 61 |
| SDSNGYVIDENGIDFDLLVDVKEKRRARLTEYAAEKATATYH | 62 |
| YAGNYDIALPCATQNEINGEAAKRLV | 63 |
| YGPAKAANAGGVAVSALEMSQNSLRLSWTREEVD | 64 |
| RLKDIMTNIFNTAKTTSETYGLDKDYLAGANIAAFENVANAMIA | 65 |
| QG | |
| NDFEAEVAATMAAALNN | 66 |
| LEDGQVLLVENTRYEDVDG | 67 |
| DKAGKSLAPVAADLAAKLGQDVVFPGVTRGAELEAAIN | 68 |
| KESKNDPELGKYWASLGDGIF | 69 |
| DAVAVEAEFAA | 70 |
| ETQADSIEEIVEVVEGDNA | 71 |
| RLEKFLGGIEDMPRIPDVMYVVDPHKEQIAVKEAKKLGIPVVAMV | 72 |
| DTN | |
| DPDDIDVIIPANDDAIRAVKLITAKLADAIIEGRQG | 73 |
| IRRNEELANSG | 74 |
| LSRKDDEGQDGPIVDYIL | 75 |
| LDVTAKYQQAVRDIVALA | 76 |
| PGTDATIQALLPHL | 77 |
| QDGPIVDYIL | 78 |
| VHASKVLPKPAAF | 79 |
| GDKAELIEK | 80 |
| AQLRVASKENNWNLPFAD | 81 |
| VPVPTFSAAITYFDSYRSAD | 82 |
| RKDKEGTFHYSW | 83 |
| ESRGYTVAIYNRSKEKTEDVIACHPEKNFVPSYDVES | 84 |
| DVLEEISAKAPEDGKPCVTYIGPDGA | 85 |
| AESYDLMQHLLGLSAEDMAEIFT | 86 |
| MNAIQESFT | 87 |
| KLFANYEANVKYQAIENAASHNGIFAALE | 88 |
| KWKVENSWGDKVGTDGYFVASDAWMDEYTYQIVVRKELLTAEEQA | 89 |
| AYGAE | |
| SFKYYVIEVNP | 90 |
| GVHHNEIPELA | 91 |
| RTDIQKIMVIGSGPIIIGQAAEFDYAGTQAC | 92 |
| SLKEEGYEVVLVNSNPATIMTDKEIADKVYIEPITLEFVTRILR | 93 |
| KE | |
| PDALLPTLGGQTGLNMAMELSKNGILDEL | 94 |
| SAIDQAEDRDLFKQLMEELEQPIPESEIVNTVEEA | 95 |
| QTMSDYENQMLRDASLSIIRALKIEGGCNVQLALDP | 96 |
| TAKLNGFSDRKIAELWGTTDD | 97 |
| VRQLRLENKIVPVYKMVDTCAAEFDSETPYFYSTYGWENESI | 98 |
| SDKESVLVLGSGPIRIGQGVEFDYATVHSVKAIQAAGYEAI | 99 |
| VLIPGYVIEVNPRASRTVPFLSKVTNI | 100 |
| EELRQEVKE | 102 |
| MTTNRLQVSLPGLDLKNPIIPASGCFGFGQEYAKYYDL | 103 |
| EAALEMYLAGASAIGVGTANFTNPYACP | 104 |
| AAQMDGAILVVAST | 105 |
| QERILEDINLQVTSGEVVS | 106 |
| IVSEIKLDWSEDEDKEVQKIAYKRQILAELGLD | 107 |
| LLKEFQELETFADF | 108 |
| AKLAEFELAGKP | 109 |
| LSGVPEAKDK | 110 |
| YKEWGMPAH | 111 |
| FLPFGVSPDFMDARINVLWASAPSTILPDTTYYAEEHPQREEL | 112 |
| TLWKESSANLLKAYDFSDEEIEDLLEKRLELDRRV | 113 |
| WSLLKATLILSVVNLSTSYLTEDIRVL | 114 |
| YQRVYRYLDKRVD | 115 |
| LKDSRTAKYHKRLQIVLF | 116 |
| ILDACPLVLDCRVDRIVEEDGICHIFAKILERLVAPE | 117 |
| LDEKGHFKNQLFAPTYFMGDG | 118 |
| MNIIEEIMTKLREDIRNIAII | 119 |
| VDELLKQSETLDARTELAERAMDSNDIEKERGITI | 120 |
| and | |
| EVVDEVLELFIELGADDDQLDFPVVYASAINGTSSLSDDPADQE. | 121 |

42. The peptide according to claim 41 consisting of 9-20 amino acids and selected from SEQ ID NOS: 26, 27, 28, 33, 34, 35, 38, 39, 42, 43, 46, 48, 53, 55, 56, 57, 66, 67, 70, 71, 74, 75, 76, 77, 78, 79, 80, 81, 82, 83, 87, 90, 91, 101, 102, 105, 106, 108, 109, 110, 111, 115 and 116, and variants and analogs thereof.

43. The peptide according to claim 41 consisting of 21-50 amino acids and selected from SEQ ID NOS: 29, 30, 31, 32, 36, 37, 40, 41, 44, 45, 47, 49, 50, 51, 52, 54, 58, 59, 60, 61, 62, 63, 64, 65, 68, 69, 72, 73, 84, 85, 86, 88, 89, 92, 93, 94, 95, 96, 97, 98, 99, 100, 103, 104, 107, 112, 113, 114, 117, 118, 119, 120, 121, and variants and analogs thereof.

44. The peptide according to claim 41 conjugated or fused to a carrier protein or to at least one carbohydrate moiety.

45. The conjugate according to claim 44 wherein the carbohydrate moiety is an S. pneumoniae capsular polysaccharide.

46. A peptide-multimer comprising a plurality of S. pneumoniae-derived peptides according to claim 41 and consisting of a maximum of 900 amino acid residues.

47. The peptide-multimer according to claim 46 comprising a plurality of repeats of at least two different peptides.

48. The peptide-multimer according to claim 47 wherein at least two peptides are derived from different S. pneumoniae cell wall or cell membrane proteins associated with an age-dependent immune response.

49. The peptide multimer according to claim 48 having a sequence set forth in any one of SEQ ID NOS: 122, 124, 126, 128 and 130.

50. The peptide-multimer according to claim 46 conjugated or fused to a carrier protein, or expressed as part of a carrier protein, or conjugated to at least one carbohydrate moiety.

51. The conjugate according to claim 50 wherein the carbohydrate moiety is an S. pneumoniae capsular polysaccharide.

52. An isolated polynucleotide sequence encoding a peptide multimer according to claim 46.

53. The isolated polynucleotide according to claim 52 having a sequence set forth in any one of SEQ ID NOS: 123, 125, 127, 129 and 131.

54. A vector comprising a polynucleotide sequence according to claim 52 operably linked to one or more transcription control elements.

55. A host cell comprising the vector of claim 54.

56. A vaccine for protecting a subject against S. pneumoniae comprising at least one peptide-multimer according to claim 46 and optionally, at least one adjuvant and/or an excipient.

57. The vaccine according to claim 56 further comprising at least one additional antigenic S. pneumoniae molecule.

58. The vaccine according to claim 57 wherein the at least one additional antigenic S. pneumoniae molecule is an S. pneumoniae polysaccharide.

59. A method for inducing an immune response and conferring protection against S. pneumoniae in a subject, wherein the method comprises administering to the subject a vaccine according to claim 56.

60. The method according to claim 59 wherein the route of administration of the vaccine is selected from intramuscular, intranasal, oral, intraperitoneal, subcutaneous, topical, intradermal, and transdermal delivery.
